(19) United States
(12) Patent Application Publication
(10) Pub. No.: US 2026/0159869 A1
(43) Pub. Date: Jun. 11, 2026

(54) DEMINERALISATION OF ORGANIC TISSUE

(71) Applicant: Marine Bioenergy AS, Skogsvåg (NO)

(72) Inventors: Arnstein Haugen, Lysekloster (NO); Torstein Frantzen, Bergen (NO); Eirik Elje, Ålgård (NO); Andrew Charles Dustan, Os (NO); Bjørn Liaset, Rådal (NO); Kjartan Sandnes, Haugsvær (NO)

(21) Appl. No.: 18/707,510

(22) PCT Filed: Nov. 4, 2022
(86) PCT No.: PCT/EP2022/080863

§ 371 Date: May 3, 2024

(30) Foreign Application Priority Data
Nov. 5, 2021 (GB) 2115923.1

(51) Int. Cl. C12P 21/06

(52) U.S. Cl. CPC C12P 21/06

(57) ABSTRACT

A method of processing an organic feedstock comprising mineralised tissues comprise: carrying out enzymatic hydrolysis of the organic feedstock to produce hydrolysis fractions and mineralised tissue remnants, wherein the mineralised tissue remnants comprise a proteinaceous portion; separating the mineralised tissue remnants from the hydrolysis fractions; and demineralising the mineralised tissue remnants to produce solubilised minerals and demineralised tissue remnants.

The present invention relates to a method of processing an organic feedstock comprising mineralised tissues, a processing plant for processing an organic feedstock comprising mineralised tissue material, and a method of demineralising mineralised tissues.

Mineralised tissues are biological tissues comprising minerals incorporated into organic matrices. Mineralised tissues typically provide some form of structural support for an organism; examples include bone, fish scales, tendons, cartilage, tooth enamel, dentin, and hooves. The organic matrices typically comprise proteins, and so form a proteinaceous portion of the mineralised tissue. Taking bone as an example of a mineralised tissue, the organic matrix makes up about 30 to 35% of the bone mass, and is referred to as ossein. Ossein comprises approximately 95% collagen (in the form of flexible elastic fibres), which is a protein. The mineral bound into the ossein matrix is predominantly calcium phosphate, in a chemical arrangement known as calcium hydroxyapatite. The ossein matrix is flexible, but the mineralisation of the matrix gives bone its rigidity.

Within the biorefining industry there is a need to increase the percentage yield of the useful end-products obtained from biomass feedstock in order to produce greater quantities of useful products more efficiently and reduce the associated waste.

Due to the increased value of meat and meat derivatives, there is a trend to scrape or mechanically separate the flesh from offcuts of animal products before they are used in biorefining processes. This reduces the quality of the feedstock available for biorefining due to the loss of proteinaceous material. In particular the removal of flesh from the offcuts increases the proportional mineral content of the offcuts leading to an undesirable increase in the proportion of ash in the final product. It is therefore desirable to create a process which is able to obtain desirable end-products using this lower quality feedstock. Here, ash is the total mineral content, determined gravimetrically (i.e. by weighing) the sample before and after heating to 550° C. for a certain period of time. The high temperature burns off all organic materials, however minerals are not destroyed by this temperature.

One example of a process used in biorefining is hydrolysis. In this process, chemical bonds in a molecule are broken by the addition of water, typically with an enzyme acting as a catalyst for the reaction. As an example, proteins can be processed by hydrolysis to obtain a wide array of peptides all the way down to single amino acids-depending on the enzymes used and the processing conditions.

It is therefore desirable to create a process which is able to produce proteins, polypeptides and amino acids of sufficient quality, particularly using lower-quality feedstock including mineralised tissues.

It has been recognised by the present inventors that removing the mineralised portion of the feedstock can improve biorefining processes.

According to a first aspect of the invention, a method of processing an organic feedstock comprising mineralised tissues comprises: removing flesh from the mineralised tissues; and demineralising the mineralised tissue remnants to produce solubilised minerals and demineralised tissue remnants.

Removal of the flesh from the mineralised tissues may be done using a number of processes. Non-limiting examples include boiling or steaming (i.e. thermic processing), the use of microbes, the use of insects, or autolysis (without the use of additional external enzymes). One particularly advantageous process for removing flesh from the mineralised tissues is enzymatic hydrolysis. As discussed above, in enzymatic hydrolysis, chemical bonds in a molecule are broken by the addition of water, with an enzyme acting as a catalyst for the reaction. To promote this reaction, the various components of the reaction mixture (for example, the raw material comprising the molecules to be hydrolysed, water, and an appropriate enzyme) must be mixed together under appropriate reaction conditions. Enzymatic hydrolysis of organic material may for example be used to obtain a wide array of peptides all the way down to single amino acids-depending on the enzymes used and the processing conditions.

Therefore, according to a second aspect of the invention, a method of processing an organic feedstock comprising mineralised tissues is disclosed, as set out in claim 1. The method comprises: carrying out enzymatic hydrolysis of the organic feedstock to produce hydrolysis fractions and mineralised tissue remnants; separating the mineralised tissue remnants from the hydrolysis fractions; and demineralising the mineralised tissue remnants to produce solubilised minerals and demineralised tissue remnants.

Optionally, the above mentioned step of enzymatic hydrolysis of the organic feedstock is carried out in order to remove flesh from the mineralised tissues. The step of enzymatic hydrolysis of the organic feedstock may comprise the use of a protease. Optionally, the hydrolysis fractions comprise the result of enzymatic hydrolysis (for example using a protease) on the flesh, predominantly. The protein in the protein-containing mineralised tissues may be shielded from the enzymatic hydrolysis process by the minerals bound within the proteinaceous matrix, and so may not be readily hydrolysed.

The following features may be combined with each of the first and second aspects as appropriate.

Optionally, the mineralised tissue remnants comprise a proteinaceous portion (i.e. a portion consisting of protein) and a mineral portion. The mineral portion and proteinaceous portion may together comprise the majority of the mineralised tissue remnants.

Optionally, the demineralised tissue remnants comprise a proteinaceous portion, wherein for example the majority of the demineralised tissue remnants comprises protein.

The organic feedstock may be derived from or comprise meat offcuts, including for example fish offcuts (for example salmon or cod), or poultry offcuts, or other animal derivatives. In particular, the organic feedstock may be derived from waste produced by the food industry, or may be a secondary product of another industrial process. The organic feedstock may comprise “industrial fish”, i.e. raw materials used for fish meal production. The processing may therefore aid in utilisation of discarded products and minimisation of waste.

Depending on the organic feedstock, the mineralised tissues present in the organic feedstock may comprise one of more of: bones, fish scales, tendons, cartilage, tooth enamel, dentin, and hooves. Preferably, the mineralised tissue comprises bones. The organic feedstock may comprise flesh and bones from aquatic animal origin (for example, marine animal origin) or terrestrial animal origin.

The mineralised tissues within the organic feedstock may comprise a proteinaceous portion and a mineral portion. In the case that the mineralised tissue is bone, the mineral portion of the mineralised tissues may comprise calcium hydroxyapatite. In the case that the mineralised tissue is bone, the proteinaceous portion may comprise ossein (comprising predominantly collagen).

Optionally, the processing of the organic feedstock is carried out as a continuous flow (i.e. non-batch) process.

The reference to a continuous flow process is intended to cover a process where the flow of at least some of the material through the stages of the process occurs in a single pass, without batch-wise processing, with the reaction mixture entering the processing plant continuously and the product of the reaction exiting the processing plant continuously (a primary continuous flow process). Depending on the nature of the reaction there may be further raw materials added continuously partway through the process and/or products may be removed continuously partway through the process. In some cases, some of the material may be removed from the primary continuous flow process, treated in a secondary continuous flow process, and then some of the material produced in the secondary continuous flow process may be reintroduced into the primary continuous flow process.

Thus, as the hydrolysis fractions and mineralised tissue remnants flow out of the enzymatic hydrolysis process, additional organic feedstock may be fed into the beginning of the enzymatic hydrolysis process to replenish the volume of reactants within the process. Similarly the volume of reactants within the demineralisation process may be replenished by mineralised tissue remnants flowing into the demineralisation process as the solubilised minerals and demineralised tissue remnants flow out.

By using a continuous flow process (a non-batch process) the processing of the organic feedstock can become more efficient and more stream-lined than a corresponding batch-wise system would be. In general, a continuous flow process requires less downtime of the vessels used for each processing stage and the associated machinery, e.g. pumps, used for carrying out the reactions during batch changeover. Time lost during the batch changeover is also avoided. The energy input and operational personnel or equipment required for carrying out the batch changeover is similarly avoided.

The method may comprise heating the material output from the enzymatic hydrolysis process (i.e. the hydrolysis fractions and mineralised tissue remnants) to a temperature sufficient to inactivate (deactivate/denature) the enzymes. In one non-limiting example, the material may be heated to about 95° C. for about 10 minutes. The temperature to which the material should be heated (and the time for which it is held at that temperature) may be chosen appropriately, depending on the enzymes used.

Demineralisation of the mineralised tissue remnants is discussed in greater detail below. Before that discussion, a brief discussion of the processing of the hydrolysis fractions is provided.

The output from hydrolysis of the organic feedstock may comprise hydrolysis fractions and mineralised tissue remnants. Here, the hydrolysis fractions may include the following fractions:
an aqueous fraction comprising water with dissolved protein, polypeptides and amino acids (the aqueous fraction may be referred to as a hydrolysate);
a sediment fraction comprising for example insoluble proteins and other small solids (the sediment fraction may typically comprise 30-40% dry matter); and
an oil fraction.

The first two of these (the aqueous fraction and sediment fraction) may together be termed “a slurry fraction”.

The hydrolysis fractions (comprising the aqueous fraction, sediment fraction and oil fraction) may be separated into the three component fractions. For example, a three-phase decanter (optionally, a centrifugal decanter) may be used. Other separator configurations are possible—for example, a combination of a two phase decanter and one or two separators.

Each of the aqueous fraction, sediment fraction and oil fraction may be further treated once separated. For example, each may undergo further separation using for example one or more centrifuges, and/or one or more filters (for example, molecular sieves or mechanical filters).

The aqueous fraction may be dried. The sediment fraction may be dried. The aqueous fraction and sediment fraction need not be fully dried, but may be partially dried. The aqueous fraction and sediment fraction may be dried to the extent that they are in a form which is stable according to its water activity. For example, the aqueous fraction may be partially dried to form a paste, usually with above 60% dry matter content.

An evaporator may be used to partially dry the aqueous fraction (to form a paste). A dryer may be used to fully dry the aqueous fraction (to form a meal). It is generally less energy-intensive (and hence less expensive) to first partially dry the aqueous fraction (to form a paste) using an evaporator, and then to transfer the paste to a dryer to fully dry the paste. Therefore, drying the aqueous fraction may be a two-step process comprising firstly partially drying the aqueous fraction (in an evaporator) to form a paste, and then fully drying the paste (in a dryer) to form a meal.

A spray dryer may be used to form a powder from the paste. The paste received by the spray dryer should typically comprise 30-50% dry matter, somewhat depending on particle size and viscosity. Where the spray dryer is not on the same site as the evaporator, the evaporator should produce a paste with over 60% dry matter (for the paste to be microbiologically stable), and then water must be added back into the paste before injection into the spray dryer.

Correspondingly, the sediment fraction also may be partially or fully dried. Since the sediment fraction has physical characteristics making it difficult to process in a traditional evaporator, a dryer may be used to partially or fully dry the sediment fraction.

Generally, a paste comprises above 60% dry matter content, and a meal comprises above 92% dry matter content.

Optionally, demineralising the mineralised tissue remnants comprises treating them with an acid solution in order to solubilise the minerals. The acid solution will dissolve the ions of the mineralised portion of the mineralised tissue remnants leaving the proteinaceous portion of the mineralised tissue remnants in the form of demineralised tissue remnants.

Optionally, the acid solution comprises hydrochloric acid (HCl).

The acid solution may alternatively or additionally comprise other strong acids. Examples of such strong acids include sulphuric acid (H₂SO₄), nitric acid (HNO₃) and phosphoric acid (H₃PO₄). In general, the stronger the acid the more effective it will be at demineralising the mineralised tissue remnants.

It has been found that HCl has an advantageous dissociation constant and is more economically viable than other acids. The use of HCl is also more practically viable and has lower risks and associated costs relating to the health and safety of using strong acids.

Optionally, the acid solution comprises acid at a concentration of between 1 and 5 wt %.

Optionally, the concentration of acid is between 1 and 4 wt %, or between 2 and 3 wt %. Preferably the concentration of acid is approximately 3 wt %.

The concentration of acid may depend on the composition of the mineralised tissue remnants and hence the type of organic feedstock that is used. The concentration of acid may also depend on the type of acid that is used. The concentration of acid should be high enough so that the rate of the demineralisation reaction is not unduly inhibited by a lack of an acidic reactant, but should also not be so high that unreacted acid is wasted. Both of these factors increase the economic efficiency of the demineralisation process.

Optionally, the weight ratio of mineralised tissue remnants to acid solution is between 1:2 and 1:8. Optionally, the weight ratio of mineralised tissue remnants to acid solution is between 1:3 and 1:5. Optionally, the weight ratio of mineralised tissue remnants to acid solution is approximately 1:5. Thus, there is a greater weight of acid solution compared to the weight of mineralised tissue remnant. This ensures that the acid solution covers all of the mineralised tissue remnants during the demineralisation so that the rate of reaction is not limited by a lack of contact between the acid and the mineralised tissue remnants. The weight proportion of acid solution is limited for spatial and economic efficiency and to limit the amount of acid solution which remains unreacted (which can then be considered to be wasted). The lower the proportion of acid solution used, the greater the volume of mineralised tissue remnants that can be processed within the same volume of container.

Optionally, demineralising the mineralised tissue remnants comprises progressive addition of acid for controlling the demineralisation rate. Thus, the concentration of reactive acid may be controlled during the demineralisation process. Reactive acid is the acid not yet consumed following a reaction with the minerals of the mineralised tissue remnants, which still has the potential to cause a reaction with the minerals in order to separate the mineral from the proteinaceous portion of the mineralised tissue remnants. The acid will be consumed as it reacts with and solubilises the mineral content of the mineralised tissue remnants. In order to maintain a desired reaction rate additional acid solution can be added during the demineralisation process. This can decrease the length of time taken to achieve the desired level of demineralisation.

Optionally, the demineralisation of the mineralised tissue remnants is carried out for between 30 to 180 minutes. Optionally, the demineralisation of the mineralised tissue remnants is carried out for between 60 to 120 minutes. Optionally, the demineralisation of the mineralised tissue remnants is carried out for approximately 60 minutes.

When the demineralisation process is carried out in a continuous flow process, the length of time for which demineralisation is carried out is dependent on the residence time of the mineralised tissue remnants within the demineralisation reaction vessel. Within the demineralisation reaction vessel, the reactants generally traverse a path moving in the direction from the inlet to the outlet. The residence time of the mineralised tissue remnants within the reaction vessel can be controlled by varying the length of the path between the inlet and outlet of the reaction vessel which the mineralised tissue remnants must travel. Hence the length of the reaction vessel may affect the duration of the demineralisation process. The speed at which the mineralised tissue remnants travel through the reaction vessel may also affect the duration of the demineralisation process. The length of time of the demineralisation process should be sufficient for substantially all of the mineral portion of the mineralised tissue remnants to be solubilised, but not so long that energy is wasted by maintaining completely demineralised tissue remnants in the demineralisation reaction vessel unnecessarily.

Optionally, the demineralisation of the mineralised tissue remnants is carried out at a temperature of between 30 and 70 degrees centigrade. Optionally, the demineralisation of the mineralised tissue remnants is carried out at a temperature of between 40 and 55 degrees centigrade. Optionally, the demineralisation of the mineralised tissue remnants is carried out at a temperature of approximately 40 degrees. Thus the temperature at which the demineralisation is carried out is advantageously elevated so as to provide energy to the reactants for an increased reaction rate, without causing damage to the reactants or products and without wasting energy or other resources by raising the temperature unnecessarily high.

Optionally, the demineralised tissue remnants undergo a further enzymatic hydrolysis process. The demineralised tissue remnants, being formed by demineralisation of the mineralised tissue remnants comprising a proteinaceous portion, may also comprise a proteinaceous portion. The enzymatic hydrolysis of the demineralised tissue remnants result in at least part of the proteinaceous portion of the demineralised tissue remnants being hydrolysed. Optionally, the enzymatic hydrolysis of the demineralised tissue remnants comprises the use of a protease.

This second hydrolysis process can have an enhanced effect compared to the effect of hydrolysis on the mineralised tissue—the demineralised tissues are more readily hydrolysed (compared to mineralised tissues) due to the lack of minerals acting as a barrier to the reaction between the proteins and the enzymes. The yield of protein/polypeptide/amino acid from the organic feedstock can therefore be increased. The mineralised tissue remnants comprise a proteinaceous portion for which it is possible, but not readily achievable, to treat via a further enzymatic hydrolysis whereby protein is broken down into its hydrolysed components. The demineralised tissue remnants may comprise a proteinaceous portion for which it is readily achievable to treat via a further enzymatic hydrolysis whereby protein is broken down into its hydrolysed components.

The method may comprise heating the material output from the further enzymatic hydrolysis process to a temperature sufficient to inactivate (deactivate/denature) the enzymes. In one non-limiting example, the material may be heated to about 95° C. for about 10 minutes. The temperature to which the material should be heated (and the time for which it is held at that temperature) may be chosen appropriately, depending on the enzymes used.

In any of the foregoing processes, or in any of the processes described below, the solubilised minerals may be separated from the demineralised tissue remnants. The solubilised minerals may be collected for further processing and/or further use, for example for use in nutritional supplements or fertilisers.

The processes may further comprise recovering the solubilised minerals, for example by neutralisation. The solubilised minerals may be neutralised using sodium hydroxide (NaOH) or potassium hydroxide (KOH) for example. The neutralised solution may be centrifuged or filtered such that precipitated minerals are collected to form a mineral slurry or mineral solids.

The mineral slurry may typically comprise calcium phosphate (CaPO₄) slurry. The neutralisation process is used to recover CaPO₄ solids for reuse, for example as a component in nutritional supplements or fertilizer. This maximises the use of the raw material by using both the proteinaceous portion and the mineral portion, thereby limiting the overall waste of the process. By centrifuging the neutralised solution, the precipitated minerals can be effectively collected as the centrifugal force aids in the separation of the solids from the liquid.

Alternatively, the mineral slurry can be collected from the neutralised solution using a filter press such as a plate and frame filter press, a membrane filter press, an automatic filter press or a recessed plate filter press, or using a drum filter such as a rotary vacuum drum filter.

In any of the foregoing processes, or in any of the processes described below, the demineralised tissue remnants may be dried. Drying the demineralised tissue remnants allows for a protein-rich product to be collected, comprising the protein making up the proteinaceous portion of the mineralised tissue remnants, which may be utilised advantageously in further processes or products. In the case that the mineralised tissue remnants comprise bone, the protein-rich product comprises predominantly collagen.

An evaporator may be used to partially dry the demineralised tissue remnants (to form a paste). A dryer may be used to fully dry the demineralised tissue remnants (to form a meal). Drying the demineralised tissue remnants may be a two-step process comprising firstly partially drying the demineralised tissue remnants (in an evaporator) to form a paste, and then fully drying the paste (in a dryer) to form a meal.

Generally, a paste comprises above 60% dry matter content, and a meal comprises above 92% dry matter content

Nitrogen is present in the products of the hydrolysis reaction mostly from the amino acids present in proteins and peptides. The protein content of the collected products can hence be determined by measuring the nitrogen content. Typically a crude protein value is used to demonstrate the protein content of a sample, this value is calculated by multiplying the nitrogen content (in g/100 g or %) (analysed using the Kjeldahl method or by combustion analysis following the Dumas principle) by a factor of 6.25.

The following discussion sets out five examples of processes which are used to target different end products.

Optionally, in a first process the method comprises dividing the mineralised tissue remnants into a first portion which is not to be demineralised and a second portion to be demineralised; separating the solubilised minerals from the demineralised tissue remnants following demineralisation of the second portion of the mineralised tissue remnants; and drying the demineralised tissue remnants together with the first portion of mineralised tissue remnants.

Thus, a first portion of the mineralised tissue remnants may be dried without undergoing a demineralisation process. The size of the first portion may be chosen so as to target a specific ash content in the end product. The motives for this may for example be related to economics (yield) or product quality (for example, calcium content, or total ash content).

Dividing the mineralised tissue remnants into a first portion and a second portion may be achieved using a diverter. The diverter may be provided by the combination of a screw conveyor and a rotary dosing valve. The mineralised tissue remnants may be moved along the screw conveyor (optionally with the screw conveyor arranged substantially horizontally, or at a small angle to the horizontal) with the rotary dosing valve attached below. A portion of the mineralised tissue remnants may be diverted out of the main flow by the rotary dosing valve, while the remainder of the mineralised tissue remnants are transported further by the conveyor. The diverter thus splits the flow of mineralised tissue remnants into two portions in a non-selective way. The relative size of the two split flows may be set by the rotational speed of the rotary dosing valve.

Optionally, in a second process the method comprises separating the solubilised minerals from the demineralised tissue remnants; and carrying out a second enzymatic hydrolysis process on the demineralised tissue remnants. The demineralised tissue remnants undergo a further enzymatic hydrolysis process where the hydrolysis can have an enhanced effect compared to the effect of hydrolysis on the mineralised tissue—the demineralised tissues are more readily hydrolysed (compared to mineralised tissues) due to the lack of minerals acting as a barrier to the reaction between the proteins and the enzymes. The yield of protein/polypeptide/amino acid from the organic feedstock can therefore be increased. By using a separate processing stage, i.e. reaction vessel, for the hydrolysis of the demineralised tissue remnants, the parameters of the reaction can be more specifically tailored to the demineralised tissue remnants, compared to for example feeding the demineralised tissue remnants back into the organic feedstock for hydrolysis process in the first hydrolysis stage.

By removing the mineral portion the protein fraction of the end product is increased, meaning the end product is purer and more refined due to a reduced mineral content. This means that further refining to reduce the ash content may no longer be required to achieve a given quality of product. Because of these advantages, carrying out a demineralisation process subsequently to a hydrolysis process and prior to a further hydrolysis process allows for the use of a lower quality raw material. A lower quality raw material may be for example fish or other animal bones that have been scraped or otherwise processed to remove the flesh from the bones. The use of the present method may ensure that using this lower quality raw material is still viable without the end product falling below a required quality. Consequently the method reduces waste by making use of the lower quality raw material that may otherwise be discarded.

Optionally, in a third process the method further comprises separating the solubilised minerals from the demineralised tissue remnants; and recycling the demineralised tissue remnants back into the organic feedstock for enzymatic hydrolysis.

The demineralised tissue remnants undergo a further enzymatic hydrolysis process where the hydrolysis can have an enhanced effect compared to the effect of hydrolysis on the mineralised tissue on account of the lack of the minerals acting as a barrier to the reaction between the proteins and the enzymes. The yield of protein/polypeptide/amino acid from the organic feedstock can therefore be increased. By recycling the demineralised tissue remnants back into the organic feedstock, they can undergo a further hydrolysis reaction without the need for a further separate processing stage to be set up. This method therefore reduces the space, number of machines and reactant resources that would otherwise be required if a second hydrolysis processing stage were used.

The demineralised tissue remnants may be processed (for example by sieving) before being reintroduced to the organic feedstock so that only those remnants above a certain size are recycled. It is generally not advantageous to have too many recycles of the same material from a product quality or process control perspective.

By recycling the demineralised tissue remnants back to the start of the process, demineralised tissue remnants that are not fully hydrolysed during the second hydrolysis process may be recycled again to the organic feedstock and can undergo a third hydrolysis process, and so on until the tissues are fully hydrolysed or small enough to be removed during a process (e.g. sieving) to remove tissues smaller than a certain size. A more complete hydrolysis can therefore be carried out, increasing the yield of protein/polypeptide/amino acid further.

The discussion above relating to the advantages of carrying out a demineralisation process subsequently to a hydrolysis process and prior to a further hydrolysis process also applies here where the demineralised tissue remnants are returned to the organic feedstock for further hydrolysis.

Optionally, in a fourth process the method further comprises separating the solubilised minerals from the demineralised tissue remnants; and partially drying the demineralised tissue remnants to form protein paste.

The demineralised tissue remnants can be dried and collected in the form of a protein meal. The demineralised tissue remnants are therefore incorporated into an end product, the protein meal, without undergoing a further hydrolysis process but by being dried.

Optionally, in a fifth process the method comprises separating the solubilised minerals from the demineralised tissue remnants; carrying out a second enzymatic hydrolysis process on the demineralised tissue remnants; and separating an output of the second enzymatic hydrolysis process into a second sediment fraction and a second aqueous fraction. The method may comprise drying the second sediment fraction. The method may comprise evaporating liquid from the second aqueous fraction to form a peptide paste; and optionally, spray-drying a portion of the peptide paste to form a peptide powder.

The solubilised minerals may be separated from the demineralised tissue remnants, that is, the solubilised minerals may be drained from a vessel containing the solubilised minerals and the demineralised tissue remnants. The solubilised minerals may be collected and can be advantageously utilised in further processes or products, or they may be disposed of.

In the fifth process, the demineralised tissue remnants undergo a further enzymatic hydrolysis process where the hydrolysis can have an enhanced effect compared to the mineralised tissue on account of the lack of the minerals acting as a barrier to the reaction between the proteins and the enzymes. The yield of protein/polypeptide/amino acid from the organic feedstock can therefore be increased.

The second sediment fraction, that is the non-soluble proteins, of the product of the second hydrolysis process can be sent to a dryer for collection. This dryer may also receive products of the primary continuous flow process and hence the second sediment fraction may join the first sediment fraction (produced by the first hydrolysis process) in the dryer for collection. This collection of the sediment fraction ensures that no product loss occurs from non-soluble proteins.

The aqueous fraction of the product of the second hydrolysis reaction is sent for further processing. The further processing may include evaporating the volatile components of the aqueous fraction, e.g. the water, leaving behind any protein/polypeptide/amino acid that was present in the aqueous fraction. The evaporated portion is drained for collection or disposal, and the remaining components either collected as a peptide paste, or spray dried to form a peptide powder. The peptide paste may be a collagen-peptide paste (i.e. a paste comprising peptides derived from the hydrolysis of collagen) and the peptide powder may be a collagen-peptide powder (i.e. a powder comprising peptides derived from the hydrolysis of collagen).

First to fifth processes have been described above, but it should be recognised that methods of processing the materials produced in the hydrolysis and demineralisation processes are not limited to use in the particular first to fifth processes described above, but may be combined in any combination so as to achieve whatever end-products are desirable.

The method of the second aspect comprises carrying out enzymatic hydrolysis of the organic feedstock to produce hydrolysis fractions and mineralised tissue remnants; separating the mineralised tissue remnants from the hydrolysis fractions; and demineralising the mineralised tissue remnants to produce solubilised minerals and demineralised tissue remnants.

Then, any of the following process steps may be combined with the foregoing method of the second aspect in any combination, so as to achieve whatever end-products are desirable.

The hydrolysis fractions may be separated into: an aqueous fraction comprising water with dissolved protein, polypeptides and amino acids; a sediment fraction comprising insoluble proteins and other small solids; and an oil fraction.

The aqueous fraction (or a portion thereof) may be dried, at least partially.

The sediment fraction (or a portion thereof) may be dried, at least partially.

The solubilised minerals may be separated from the demineralised tissue remnants.

The solubilised minerals may be recovered by neutralising the solution of solubilised minerals is neutralised, optionally using NaOH or KOH. The neutralised solution may be centrifuged or filtered such that minerals are collected to form a mineral slurry. The mineral slurry may be dried, at least partially, to form mineral solids.

The mineralised tissue remnants (or a portion thereof) may be dried, at least partially.

The demineralised tissue remnants (or a portion thereof) may be dried, at least partially.

Mineralised tissue remnants (or a portion thereof) and demineralised tissue remnants (or a portion thereof) may be dried (at least partially) together.

A second enzymatic hydrolysis process may be carried out on at least a portion of the demineralised tissue remnants. The second enzymatic hydrolysis process may comprise the use of a protease. The resulting aqueous fraction (comprising water with dissolved protein, polypeptides and amino acids) and sediment fraction (comprising insoluble proteins and other small solids) may each be dried (at least partially).

At least a portion of the demineralised tissue remnants may be recycled back into the organic feedstock for enzymatic hydrolysis.

In any of the foregoing processes, optionally demineralising the mineralised tissue remnants takes place in a rotating drum reactor, wherein the rotating drum reactor comprises: a drum; an inlet at a first point on the drum; a screw within the drum, wherein the screw includes a helical blade extending along the length of the drum with the outer edge of the helical blade being fixed to the inner surface of the drum such that material can be conveyed and mixed by the helical blade as the drum rotates; and an outlet at a second point along the drum. Optionally, the drum is arranged with the length of the drum and the axis of rotation of the drum extending broadly along the horizontal.

Optionally, the enzymatic hydrolysis of the organic feedstock takes place in a rotating drum reactor, wherein the rotating drum reactor comprises: a drum; an inlet at a first point on the drum; a screw within the drum, wherein the screw includes a helical blade extending along the length of the drum with the outer edge of the helical blade being fixed to the inner surface of the drum such that material can be conveyed and mixed by the helical blade as the drum rotates; and an outlet at a second point along the drum. Optionally, the drum is arranged with the length of the drum and the axis of rotation of the drum extending broadly along the horizontal.

Optionally, enzymatic hydrolysis of the organic feedstock takes place in a first rotating drum reactor, and demineralising the mineralised tissue remnants takes place in a second rotating drum reactor.

The rotating drum aids in mixing the reactants of the enzymatic hydrolysis or the demineralisation processes, whilst the turning of the screw aids in progressing the materials from the inlet to the outlet. The blade of the screw conveys the material lengthwise along the drum. This can produce more effective mixing and processing of materials than prior art devices that do not incorporate rotation or a screw blade.

The residence time of reactants within the rotating drum may be affected by the number of turns of the screw blade (given by the length of the rotating drum reaction vessel (in metres) divided by the pitch (in metres)) and the speed of rotation.

The ratio of the pitch to the length of the drum may for example be approximately 1:20, approximately 1:30 or approximately 1:40.

Further structural features of the rotating drum reactor are described below as optional features in connection with the fourth aspect of the present invention, and such features are also applicable here to a rotating drum used in the present method (to carry out enzymatic hydrolysis of the organic feedstock and/or demineralising the mineralised tissue remnants).

Optionally, the method further comprises grinding the organic feedstock prior to carrying out the enzymatic hydrolysis.

Thus, the organic feedstock material may be mechanically ground and separated into parts which are reduced in size prior to the enzymatic hydrolysis process. This creates a greater surface area of the organic feedstock material that can then contact the enzymatic hydrolysis reagents. With a greater area of contact between the reactants the speed of the reaction can be increased. As well as a greater surface area, the smaller parts of the organic feedstock require the hydrolysis reagent to penetrate a shorter distance in order to react with the material at the centre of each piece. Hydrolysis is therefore achieved more quickly and more efficiently than for larger pieces. Additionally, by reducing the size of the organic feedstock material, high shear forces within the reagents during hydrolysis can be avoided. Shear forces are caused by a fluid velocity that is too high, and can contribute towards the production of emulsions, which is undesirable.

Optionally, separating the mineralised tissue remnants from the hydrolysis fractions is effected using a sieve, or may be effected using dewatering screws, drum sieves, filters, a diverter or devices for separating components via settling. Where a sieve is used, the sieve may optionally be a vibrating sieve. The sieve may have a hole size of between 2 and 6 mm, for example. Multiple sieves may be used in order to separate multiple groups of particle sizes.

According to a third aspect of the invention, a processing plant for processing an organic feedstock comprising mineralised tissue material is provided, as set out in claim 21. The processing plant comprises: a first reaction vessel configured to perform enzymatic hydrolysis of the organic feedstock; a first separator configured to receive the output from the first reaction vessel, for separating mineralised tissue remnants output from the first reaction vessel from hydrolysis fractions output from the first reaction vessel; and a second reaction vessel configured to perform demineralisation of the mineralised tissue remnants.

Optionally, the mineralised tissue remnants comprise a proteinaceous portion.

Optionally, the processing plant is configured to carry out the method of the first or second aspect, optionally including any of the optional method features described above.

The processing plant is optionally configured to carry out processing of the organic feedstock as a continuous process.

The processing plant may comprise an inactivation section downstream of the first reaction vessel to heat the material output from the first reaction vessel to a temperature sufficient to inactivate the enzymes (i.e. the enzymes are deactivated, or denatured). The material may for example pass through a pipe surrounded by a heat exchanger, to heat the pipe and contents. In one non-limiting example, the material may be heated to about 95° C. for about 10 minutes. The temperature to which the material should be heated (and the time for which it is held at that temperature) may be chosen appropriately, depending on the enzymes used.

The organic feedstock may be derived from or comprise meat offcuts, including for example fish offcuts (for example salmon or cod), or poultry offcuts, or other animal derivatives. In particular, the organic feedstock may be derived from waste produced by the food industry, or may be a secondary product of another industrial process. The organic feedstock may comprise “industrial fish”, i.e. raw materials used for fish meal production. The processing may therefore aid in utilisation of discarded products and minimisation of waste.

Depending on the organic feedstock, the mineralised tissues present in the organic feedstock may comprise one of more of: bones, fish scales, tendons, cartilage, tooth enamel, dentin, and hooves. Preferably, the mineralised tissue comprises bones. The organic feedstock may comprise flesh and bones from aquatic animal origin (for example, marine animal origin) or terrestrial animal origin.

The mineralised tissues within the organic feedstock may comprise a proteinaceous portion (i.e. a portion consisting of protein) and a mineral portion. The mineral portion and proteinaceous portion may together comprise the majority of the mineralised tissue remnants. In the case that the mineralised tissue is bone, the mineral portion of the mineralised tissues may comprise calcium hydroxyapatite. In the case that the mineralised tissue is bone, the proteinaceous portion may comprise ossein (comprising predominantly collagen).

Optionally, the demineralised tissue remnants comprise a proteinaceous portion, wherein for example the majority of the demineralised tissue remnants comprises protein.

Optionally, the processing plant comprises a sieve, a dewatering screw press, a filter, or a density separator for separating the mineralised tissue remnants from the hydrolysis fractions.

Optionally, the processing plant comprises a three-phase separator for separating the hydrolysis fractions into: an aqueous fraction comprising water with dissolved protein, polypeptides and amino acids; a sediment fraction comprising insoluble proteins; and an oil fraction.

Optionally, the processing plant comprises a separator for separating the solubilised minerals from the demineralised tissue remnants.

Optionally, the processing plant comprises a third reaction vessel for carrying out a second enzymatic hydrolysis process on a portion of the demineralised tissue remnants. Thus the processing plant is configured to carry out enzymatic hydrolysis of the demineralised tissue remnants output from the second reaction vessel. The enzymatic hydrolysis of the demineralised tissue remnants will result in at least part of the proteinaceous portion of the demineralised tissue remnants being hydrolysed. The mineralised tissue remnants comprise a proteinaceous portion for which it is possible, but not readily achievable, to treat via a further enzymatic hydrolysis whereby protein is broken down into its hydrolysed components. The demineralised tissue remnants may comprise a proteinaceous portion for which it is readily achievable to treat via a further enzymatic hydrolysis whereby protein is broken down into its hydrolysed components. Optionally, the second enzymatic hydrolysis process comprises the use of a protease.

In this example, separate vessels are used to carry out the enzymatic hydrolysis of the organic feedstock and the enzymatic hydrolysis of the demineralised tissue remnants. The reaction parameters, for example the temperature, duration, selection of enzymes (for example, a protease) to be used, concentration of reagents within the enzymatic hydrolysis solution and ratio of hydrolysis solution to solids, can differ within each reaction vessel and can therefore be tailored to the requirements of the reacting materials.

The processing plant may comprise an inactivation section downstream of the third reaction vessel to heat the material output from the third reaction vessel to a temperature sufficient to inactivate the enzymes (i.e. the enzymes are deactivated, or denatured). The material may for example pass through a pipe surrounded by a heat exchanger, to heat the pipe and contents. In one non-limiting example, the material may be heated to about 95° C. The temperature to which the material should be heated may be chosen appropriately, depending on the enzymes used.

Optionally, the processing plant comprises a separator for separating an output of the third reaction vessel into a sediment fraction and an aqueous fraction.

Optionally, the first reaction vessel is configured to receive a portion of the demineralised tissue remnants output from the second reaction vessel.

Optionally, the processing plant comprises one or more dryers, wherein the one or more dryers comprise: a mill dryer and/or an evaporator, and/or a spray dryer.

An evaporator may be used to partially dry the aqueous fraction (to form a paste). A dryer may be used to fully dry the aqueous fraction (to form a meal). It is generally less energy-intensive (and hence less expensive) to first partially dry the aqueous fraction (to form a paste) using an evaporator, and then to transfer the paste to a dryer to fully dry the paste. Therefore, drying the aqueous fraction may be a two-step process comprising firstly partially drying the aqueous fraction (in an evaporator) to form a paste, and then fully drying the paste (in a dryer) to form a meal.

A spray dryer may be used to form a powder from the paste. The paste received by the spray dryer should typically comprise 30-50% dry matter, somewhat depending on particle size and viscosity. Where the spray dryer is not on the same site as the evaporator, the evaporator should produce a paste with over 60% dry matter (for the paste to be microbiologically stable), and then water must be added back into the paste before injection into the spray dryer.

Correspondingly, the sediment fraction also may be partially or fully dried. Since the sediment fraction has physical characteristics making it difficult to process in a traditional evaporator, a dryer may be used to partially or fully dry the sediment fraction.

Optionally, the processing plant comprises a diverter configured to split a flow of mineralised tissue remnants or demineralised tissue remnants into a plurality of flows. The diverter may be provided by the combination of a screw conveyor and a rotary dosing valve. The mineralised tissue remnants may be moved along a screw conveyor (with the screw conveyor arranged substantially horizontally, or at a small angle to the horizontal) with the rotary dosing valve attached below. A portion of the mineralised tissue remnants may be diverted out of the main flow by the rotary dosing valve, while the remainder of the mineralised tissue remnants are transported further by the conveyor. The diverter thus splits the flow of mineralised tissue remnants into two portions in a non-selective way. The relative size of the two split flows may be set by the rotational speed of the rotary dosing valve.

Optionally, at least one of the first, second and third reaction vessels is a rotating drum reactor, wherein the rotating drum reactor comprises: a drum which is rotatable about a central longitudinal axis of the drum; an inlet at a first point on the drum; a screw within the drum, wherein the screw includes a helical blade extending along the length of the drum with the outer edge of the helical blade being fixed to the inner surface of the drum such that material can be conveyed and mixed by the helical blade as the drum rotates; and an outlet at a second point along the drum. Optionally the central longitudinal axis of the drum is substantially horizontal.

The foregoing optional features of a processing plant may be combined in any combination in order to arrive at a processing plant suitable for producing the desired end products. The following discussion sets out five examples of processing plants which are used to target different end products.

Optionally, in a first processing plant the processing plant further comprises a diverter and a dryer, wherein the diverter is configured to divert a first portion of the mineralised tissue remnants output from the first reaction vessel to the dryer, and to divert a second portion of the mineralised tissue remnants output from the first reaction vessel to the second reaction vessel.

The dryer may be a mill dryer which simultaneously grinds and dries the mineralised tissue remnants.

Any desired proportion of the mineralised tissue remnants may be diverted to the dryer depending on the relevant factors of the process, for example, the raw materials used, the products desired to be collected and the process economics. The appropriate percentages of proportion sent to the dryer may be chosen accordingly.

The second portion of the mineralised tissue remnants may comprise the remaining portion of the mineralised tissue remnants after the first portion is removed.

Optionally, the dryer is also configured to receive demineralised tissue remnants output from the second reaction vessel.

The dryer removes remaining water or other liquid from the first portion of the mineralised tissue remnants output from the first reaction vessel, and where appropriate removes remaining water or other liquid from the demineralised tissue remnants output from the second reaction vessel.

Optionally, a second dryer may receive the demineralised tissue remnants output from the second reaction vessel.

Optionally, in a second processing plant the processing plant further comprises: a third reaction vessel configured to perform enzymatic hydrolysis of demineralised tissue remnants output from the second reaction vessel.

In this example, separate vessels are used to carry out the enzymatic hydrolysis of the organic feedstock and the enzymatic hydrolysis of the demineralised tissue remnants. The layout of the processing plant can be more flexible in this configuration than if each of the enzymatic hydrolysis processes are to be carried out in the same reaction vessel since there is less constraint on how the vessels must be positioned to form such a loop.

Optionally, in a third processing plant the first reaction vessel is configured to receive demineralised tissue remnants output from the second reaction vessel.

Hence the demineralised tissue remnants output from the second reaction vessel are recycled to the first reaction vessel. The demineralised tissue remnants may be combined with the raw starting material, i.e. the organic feedstock, to enter the first reaction vessel at the inlet of the first reaction vessel. This processing plant may require a smaller space to operate in compared to a processing plant in which hydrolysis of the demineralised tissues takes place in a third reaction vessel, since only two reaction vessels are required. Other resources can also be saved compared to such a processing plant including reactant resources and energy required for the reaction.

Optionally, in a fourth processing plant the processing plant further comprises: a dryer configured to dry demineralised tissue remnants output from the second reaction vessel.

The dryer is able to form protein paste from the demineralised tissue remnants output by the second reaction vessel. This protein paste may be collagen paste. Hence the demineralised tissue remnant may not undergo a second enzymatic hydrolysis reaction before it is collected and used to form a product.

Optionally, in a fifth processing plant the processing plant further comprises: a third reaction vessel configured to perform enzymatic hydrolysis (for example using a protease) of demineralised tissue remnants output from the second reaction vessel; a second separator configured to receive the output of the third reaction vessel, for separating a sediment fraction of the output of the third reaction vessel from an aqueous fraction of the output of the third reaction vessel; an evaporator configured to dry the aqueous fraction of the output of the third reaction vessel to form a peptide paste; and a spray dryer configured to form a peptide powder from the peptide paste. The paste received by the spray dryer should typically comprise 30-50% dry matter, somewhat depending on particle size and viscosity. Where the spray dryer is not on the same site as the evaporator, the evaporator should produce a paste with over 60% dry matter (for the paste to be microbiologically stable), and then water must be added back into the paste before injection into the spray dryer.

Thus the demineralised tissue remnants undergo a further enzymatic hydrolysis process, hydrolysis of the demineralised tissue remnants may go further to completion than for the mineralised tissue of the organic feedstock since the minerals which may have inhibited the hydrolysis reaction have now been removed.

The sediment fraction from the third reaction vessel, that is the non-soluble proteins remaining following the enzymatic hydrolysis of the demineralised tissue remnants, can be sent to a dryer for collection. This dryer may also receive products from the first reaction vessel and hence the solid fraction from the third reaction vessel may re-join the sediment fraction from the first hydrolysis process in the dryer for collection. This collection of the sediment fraction ensures that no product loss occurs from non-soluble proteins. The aqueous fraction of the product of the second hydrolysis reaction is sent for further processing. The further processing may include evaporating the volatile components of the aqueous fraction, e.g. the water, leaving behind any protein/polypeptide/amino acid that was present in the aqueous fraction. The evaporated portion is drained for collection or disposal, and the remaining components either collected as a peptide paste, or spray dried to form a peptide powder, or the peptide paste may be sent to a dryer in order to form a peptide meal. The peptide paste may be a collagen-peptide paste (i.e. a paste comprising peptides derived from the hydrolysis of collagen) and the peptide powder may be a collagen-peptide powder (i.e. a powder comprising peptides derived from the hydrolysis of collagen).

Optionally, at least one of the first, second and third reaction vessels is a rotating drum reactor, wherein the rotating drum reactor comprises: a drum; an inlet at a first point on the drum; a screw within the drum, wherein the screw includes a helical blade extending along the length of the drum with the outer edge of the helical blade being fixed to the inner surface of the drum such that material can be conveyed and mixed by the helical blade as the drum rotates; an outlet at a second point along the drum.

The drum is optionally arranged with the length of the drum and the axis of rotation of the drum extending broadly horizontally.

Optionally, each of the first, second and third reaction vessels is a rotating drum reactor.

The rotating drum aids in mixing the reactants of the enzymatic hydrolysis or the demineralisation processes, whilst the turning of the screw aids in progressing the materials from the inlet to the outlet along the turns of the screw. The blade of the screw conveys the material lengthwise along the drum. This can produce more effective mixing and processing of materials than prior art devices that do not incorporate rotation or a screw blade.

Further structural features of the rotating drum reactor are described below as optional features in connection with the fourth aspect of the present invention, and such features may be combined with the rotating drum reactor in the case that it is optionally used as at least one of the first, second and third reaction vessels in the above described first, second or third aspects of the invention.

The processing plant according to the third aspect may be configured to carry out the method of processing an organic feedstock comprising mineralised tissues according to the first and/or second aspects.

The concept of using a rotating drum reactor for demineralisation of mineralised tissues is considered to be independently patentable.

Therefore, according to a fourth aspect of the invention, a method of demineralising mineralised tissues is provided, as set out in claim 33. The method comprises treating the mineralised tissues with an acid solution in a rotating drum reactor, the rotating drum reactor comprising: a drum; a drum inlet at a first point on the drum; a screw within the drum; and a drum outlet at a second point along the drum, wherein the screw comprises a helical blade extending along the length of the drum with the outer edge of the helical blade being fixed to the inner surface of the drum, such that material in the drum is mixed and conveyed through the drum by the helical blade as the drum rotates.

The drum is optionally arranged with the length of the drum and the axis of rotation of the drum extending broadly horizontally.

The drum of the rotating drum reactor may extend broadly horizontally so that the level of the fluid in the drum remains substantially at the same distance from the bottom of the drum at all points along the length of the drum.

The outlet of the drum may be downstream of the inlet such that reactants introduced to the drum at the inlet flow along the axial extent of the drum from the inlet to the outlet. The inlet may hence be horizontally displaced from the outlet. The inlet may be at or substantially at one end of the drum and the outlet may be at or substantially at the opposite end of the drum. The drum may be cylindrical having substantially the same axial cross section throughout its length. The axial cross section of the drum may be circular.

The radially outer edge of the helical blade may be fixed to the inner surface of the drum, the helical blade therefore tracks the internal circumference of the drum as the helix progresses from the inlet at one end of the drum to the outlet at the opposite end of the drum. The blade guides and urges the solid material at the bottom of the drum as well as the fluid in the drum towards the outlet of the drum. As the drum rotates the reactants are mixed which aids in sustaining a high reaction rate. In order that the apparatus can be used with fluid raw materials the helical blade is preferably attached at its outer edge to the internal surface of the drum with a watertight join.

With this arrangement the screw blade forms a sequence of chambers between adjacent turns of the helical blade. Here, a “turn” of the helical blade traces out a 360° rotation. Put another way, the pitch of the screw blade is the distance between any two points of the helix that are exactly one turn apart.

In some modes of operation of the rotating drum reactor, separate batches of material can be processed in each chamber without direct contact with adjacent batches of material in adjacent chambers. Advantageously, this allows for different reaction mixtures to be used in the different chambers, for example by introducing additional reactants as the material is conveyed along the length of the drum.

Optionally, the method is carried out as a continuous flow process. Hence the method is preferably not carried out in a batch process in which the demineralisation process is carried out in blocks with process down-time between each batch as the reaction products are removed and fresh reactants are added, but instead the demineralisation process is carried out such that fresh reactants are continually, or substantially continually, added at the inlet as the products are continually, or substantially continually removed from the reactor drum via the outlet. Here, the reference to a continuous flow process is intended to cover a process where the flow through the rotating drum occurs in a single pass, without repeated circuits or batch-wise processing. Depending on the nature of the reaction there may be further materials added continuously partway through the process.

Optionally, the helical blade extends from the inner surface of the drum toward the centre of the drum but does not extend along the entire diameter of the drum such that an open channel extends along the axial length of the drum. This can allow for access for maintenance as well as during manufacture, whilst also enabling easier cleaning of the device since there are fewer fully enclosed chambers. Such a configuration also allows for operation of the rotating drum reactor in the “flooded mode”, discussed in more detail below. In such a mode, the open channel may allow for the fluid to pass between the chambers where sufficient fluid is present to overflow the barrier of the chamber formed by the blade. The screw blade may extend from the wall of the drum inward for at least 50% of the radius of the drum, for example at least 60% or about 70% of the radius of the drum.

Optionally, the acid solution fills the rotating drum reactor to above the level of the helical blade. This is referred to as the “flooded mode” of operation of the rotating drum reactor.

The solid portion of the organic feedstock (comprising mineralised tissues such as bones, and other animal parts such as fish scale, hooves and the like) will sink to the base of the drum so that the solid parts remain in their separated volumes between the walls of the blade. However, in the flooded mode of operation, the acid within the chambers of the drum will overflow to the subsequent chambers in the direction of the outlet causing an increase in the flow of acid solution in the direction of the outlet of the drum. The drum, when operated in the flooded mode, therefore enables delivery of fresh acid to the chambers of the drum towards the outlet. The flow of acid solution aids in the replenishment of consumed acid in the chambers towards the outlet of the drum and hence maintains the demineralisation reaction rate along the length of the drum.

The density of the mineralised tissue ensures that it remains at the base of the chambers without flowing through the open channel via the flow caused by the acid solution overflow. The residence time of the mineralised tissue is therefore unaffected by the use of the flooded mode but instead remains controlled by the rotation of the drum.

By modifying the process to replenish reactive acid along the length of the drum, the demineralisation can be completed in a shorter time and correspondingly over a shorter length of the drum. The drum can consequently be made smaller.

Optionally, the ratio of acid solution to mineralised tissue remnants increases along the length of the drum from the drum inlet to the drum outlet.

The progressive increase in acid allows for the demineralisation rate to be controlled, for example, acid consumed via the demineralisation process occurring in chambers towards the inlet of the drum can be replenished as the reactants are conveyed towards the outlet of the drum to provide a more constant volume of reactive acid along the length of the drum and therefore approach a more constant demineralisation rate throughout the process. The addition can be empirically optimized for a given raw material and equipment configuration. By modifying the process to increase the ratio of acid solution along the length of the drum, and thereby increasing the rate of reaction relative to keeping the ratio constant, the demineralisation can be completed in a shorter time and correspondingly over a shorter length of the drum. The drum can consequently be made smaller.

The screw blade is a helical screw blade and hence describes a spiral along the inside of the rotating drum. The screw blade may have a constant pitch along the length of the drum, or there may be a change in pitch of the screw blade between the inlet and the outlet.

Optionally, the pitch of the helical blade increases along the length of the drum, from the drum inlet towards the drum outlet. Each chamber therefore increases in volume towards the outlet of the drum. This is particularly advantageous when using a drum operating in the flooded mode, or when there is addition of fluid to the material within the drum as it passes along the drum (as discussed in more detail below)—the increasing volume of the chambers can advantageously be used to accommodate the increased volume of material. Since the mineralised tissue remains at the base of the drum and separated by the walls of the blade, the volume of tissue within each chamber will remain constant as the tissue progresses through the drum. However, given that in the flooded mode all the chambers will be filled to the same level as the acid solution can overflow the walls of the blade, the volume of acid solution within each chamber increases along the length of the drum as the pitch of the helical blade increases in correspondence with the increase in volume of the chamber. The ratio of acid solution to mineralised tissue therefore increases along the length of the drum. This has the advantage of increasing the presence of reactive acid compared to consumed acid and therefore aids in the maintenance of a desired reaction rate.

As discussed above, in some embodiments the helical blade extends from the inner surface of the drum toward to the centre of the drum but does not extend along the entire diameter of the drum such that an open channel extends along the axial length of the drum. Then, the chambers formed between adjacent turns of the screw blade are open to the hole at the centre of the drum. Alternatively however, these chambers may be closed, for example by a cylindrical body along the centre of the drum in that is fixed to the inner edge of the screw blade, preferably in a watertight fashion. In this case, there is no open channel extending along the axial length of the drum. With this arrangement the screw blade forms a sequence of enclosed chambers where separate batches of material can be processed without direct contact with adjacent batches of material. Advantageously, this allows for different reaction mixtures to be used in the different chambers, for example by introducing additional reactants as the material is conveyed along the length of the drum. A configuration with such enclosed chambers can allow for greater volumes of material to be held without risk of spilling between adjacent turns of the screw blade, as well as allowing for a smaller headspace and potentially greater control of the atmosphere within the headspace. Additionally, allowing a higher degree of filling can make it possible to reduce the size of the drum.

Optionally, the rotating drum reactor comprises a plurality of fluid inlets opening into the drum along the axial length of the drum.

The fluid inlets may be arranged to supply fluid under pressure so that the fluid jets out of the fluid inlets into the material within the drum. This can aid mixing of the material.

The use of fluid inlets may allow for the ratio of materials to be adjusted, for example by adding diluents or additional reagents to the materials within the rotating drum. Preferably there are fluid inlets in sufficient numbers and with suitable spacing to allow for one or more fluid inlet(s) for each turn of the screw. In this case fluid can be added to the original raw materials during each turn of the screw thereby increasing the amount of added fluid compared to the amount of the original raw materials as the material that is being mixed passes lengthwise along the drum.

The fluid inlets may include groups of fluid inlets at locations spaced apart along the screw blade.

It is advantageous for the apparatus to be arranged so that the supply of fluid via the fluid inlets into the drum can be controlled. Thus, the apparatus may include fluid flow control devices for controlling the rate of flow of fluid through the fluid inlets and in particular for allowing and preventing flow. For example, the apparatus may include valves for controlling flow to each fluid inlet or to groups of fluid inlets. In this case it is preferred for a controller to be provided that is arranged to permit flow through fluid inlets that are immersed within the material that is being mixed, and to prevent flow when the fluid inlets are not within the material that is being mixed. Thus, flow would be enabled when the fluid inlets are at their low point in rotation of the drum and within the level of the material that is being mixed within the drum, whereas flow would not be allowed when the fluid inlets are at higher points during rotation of the drum when they are above the level of the material that is being mixed. In one example the controller is linked with sensors allowing for rotation of the drum to be monitored, such that fluid inlets are only permitted to supply fluid when they are at a position where immersion within the material to be mixed is expected. Alternatively or additionally the controller may comprise switching devices located adjacent to the expected level of material within the drum, such that individual fluid inlets are activated and deactivated as they pass the switching devices and enter or exit the material at the base of the drum.

The method may include introducing fluids into the material, for example introducing liquids or gases as mentioned above. The method may include heating or cooling the material in the drum by introducing fluid at elevated or lowered temperature. Heating or cooling the material may be done to inactivate/denature the enzymes to cause an enzymatic reaction to cease.

The method may include controlling the supply of fluid via the fluid inlets so that the fluid is only supplied when the fluid inlets are immersed in the material in the drum. This can be done using features as discussed above, for example by controlling the flow of fluid according to the position of the respective fluid inlets in the drum.

Each of the fluid inlets may be connected to pipework for supply of fluid from a source of fluid to the fluid inlet. This pipework may advantageously be located toward the centre of the drum, thereby minimising the risk of contact of the pipework with the material that is being mixed within the drum. This could cause corrosion or fouling of the pipework or of the mixture within the drum. Alternatively, the pipework may be on the outside of the drum and optionally may connect to the mixing devices by passing within the body of the screw blade, thereby avoiding any contact with the material within the drum. It is preferred for the connections allowing for passage of fluid into the drum from the source of fluid to be located at a centre of the rotating drum, for example at one end or at both ends of the drum. Accordingly, the drum may comprise a rotary valve located along the axis of rotation of the drum, wherein the rotary valve is configured to allow fluid to pass into the pipework within the drum, during rotation of the drum. Optionally two rotary valves may be provided, each at one of the two ends of the drum.

Optionally acid solution is introduced to the drum through the plurality of fluid inlets. Thus, fresh acid that is able to react with the minerals of the mineralised tissues is introduced into the drum at various points other than at the inlet. The amount of additional acid solution added to the drum may increase towards the outlet of the drum to ensure a sufficient volume of reactive acid is present to continue the demineralisation process at a desired rate. The rate of reaction may remain substantially constant.

As discussed above, the pitch of the helical blade may increase along the length of the drum, from the drum inlet towards the drum outlet. This is particularly advantageous in the case that additional acid is introduced to the drum through the plurality of fluid inlets towards the drum outlet.

The rotating drum may comprise a plurality of mixing devices for promoting mixing of the material in each of the volumes of material as the material is conveyed along the screw, wherein the plurality of mixing devices are spaced apart along the blade of the screw, and wherein there may be at least one mixing device for each turn of the screw blade.

With this arrangement, the material that is to be mixed and processed progresses from the inlet to the outlet along the turns of the screw whilst undergoing mixing due to the rotation of the drum, the “pushing” of the blade of the screw to convey the material lengthwise along the drum, and also due to the addition of mixing devices spaced apart along the blade of the screw. This can produce more effective mixing and processing of materials than prior art devices that do not include additional mixing devices at each turn of the screw. By placing the mixing devices spaced apart along the screw and having at least one mixing device for each turn of the screw then the mixing devices act on each volume of the material as it is conveyed along the screw.

It is preferred for there to be multiple mixing devices for each turn of the screw, for example there may be a sufficient number of mixing devices so that at least one of the mixing devices is always in contact with the material as it is conveyed along the screw. It will be appreciated that in this type of rotating screw device the material that is being mixed and conveyed sits in the base of the device in the chambers that are formed between the turns of the screw and there is a headspace above the level of the material. In order to obtain best effect from the mixing devices it is advantageous for there to always be a mixing device below the level of the material and contacting the material so that there is never any period when the material is not subject to the action of the mixing device. For example, if the level of the material within the drum during normal use can be approximated as a segment of a circle (assuming a cylindrical drum) formed by a chord subtending an arc of 90° then if there are four equally spaced mixing devices on each turn of the blade of the screw then there will always be a mixing device in contact with the material. There may be four or more mixing devices for each turn of the blade of the screw, optionally five or more, or six or more. A greater number of mixing devices could be included either when the level of material within the drum is expected to be low enough to require a smaller spacing between the devices during normal use, or when it is considered to be an advantage to have more than one mixing device in contact with the material at any one time.

The mixing devices may comprise mixing vanes spaced apart along the screw with multiple vanes for each turn of the screw, the vanes being arranged to promote mixing of the material to be processed.

Each of the plurality of mixing devices may comprise a mixing vane, or alternatively there may be multiple types of mixing devices of which only some of the plurality of mixing devices comprise a mixing vane. A mixing vane preferably takes the form of an element mounted to the blade of the screw with a surface having a greater angle of attack than the surface of the blade of the screw. Thus, the mixing vane may include a ramp surface with a greater angle of attack than the surface of the screw blade.

Each mixing vane may include an identical ramp surface with the same angle of attack, or alternatively the angle of ramp surface for the mixing vane may vary, for example with an increase in the angle of attack or a decrease in the angle of attack for the mixing vane depending on its position along the extent of the blade of the screw.

In one example the ramp surface of the mixing vane is an upper surface of a wedge shaped element with the lower surface of the wedge shaped element being adjacent the surface of the blade of the screw and either attached thereto or integrally formed therewith, for example it may be attached by welding or formed with the blade of the screw in a casting process. The mixing vane may be considered to have a leading-edge at the start of the ramp surface, where in use the mixing vane first enters the material held within the volume between two turns of the screw, and a trailing edge at the end of the ramp surface which is furthest from the surface of the screw blade, the trailing edge being the edge that last meets the material held within the screw. In the case where a wedge shaped element is used then the trailing edge would be the apex of the wedge shaped element at the point furthest from the surface of the screw blade.

It is advantageous for the mixing vane to be mounted at the outer part of the surface of the screw blade, i.e. adjacent to the inner wall of the drum. This means that the mixing vane will affect mixing of all of the materials in the volume between two turns of the screw, including at the deepest extent of those materials. The mixing vane may extend from the inner wall of the drum along the surface of the screw blade toward the centre of rotation of the drum. The mixing vane could extend toward the centre of rotation to the same extent as the screw blade or to a lesser extent. Except when used in the flooded mode, the screw blade would typically extend sufficiently far toward the centre of the drum so as to be above the level of the material held in each volume between turns of the screw. This avoids any intermixing of materials from adjacent turns. The mixing vane may extend toward the centre of the drum to a lesser extent than the screw blade, for example to an extent required to ensure that the mixing vane is fully immersed in the material held in the volume between two turns of the screw. The greatest impact of the mixing vane will typically be at the deepest part of the material in the volume between two turns of the screw, since this is where larger and heavier elements of the material will gather.

The height of the mixing vane, i.e. the extent of the mixing blade from the wall of the drum toward the centre of the drum may be at least 30% of the expected level of material in the drum, for example 40% of this level or more. The screw blade may typically not extend fully across the width of the drum and hence there may be a hole along the centre of the screw blade at the centre of the rotating drum. The height of the mixing vane may be at least 20% of the height of the screw blade, for example at least 30% of the height of the screw blade. It is preferred that the mixing vane is fully immersed when it is with the deepest part of the material held in the drum and hence the height of the mixing vane may be less than the expected level of the liquid, such as 80% or less of the height of the liquid. With typical levels of liquid in devices of this time this may require a vane with a height of less than 70% of the height of the screw blade, optionally less than 60% of the height of the screw blade. Possible dimensions for the screw blade discussed in more detail below.

The mixing devices may comprise fluid inlets (as described above) for the addition of fluid to the mixture within each volume between turns of the screw. Each of the plurality of mixing devices may comprise a fluid inlet, or alternatively there may be multiple types of mixing devices of which only some of the plurality of mixing devices comprise a fluid inlet. The fluid inlets may advantageously be combined with mixing vanes, optionally with the fluid inlets opening into the drum at a surface or an edge of the mixing vane. In one example each of the mixing devices comprises a mixing vane having a ramp surface as described above, and also having one or more fluid inlets at a trailing edge of the ramp surface, i.e. at the furthest point of the ramp from the screw blade.

The mixing devices may alternatively or additionally comprise one or more of: vanes, paddles, scoops or ridges in the wall of the drum, moving parts attached to the drum or the screw blade (e.g. rotors) with passive or active movement, and/or other static or dynamic mixing devices.

The inlet to the rotating drum and the outlet from the rotating drum may be at longitudinal ends of the drum. The inlet may comprise an opening at an inlet end of the drum, with an inlet pipe passing through the opening and allowing for material to be fed into the rotating drum reactor. In the case of a cylindrical drum the inlet end of the drum may comprise an enclosed outer part with an open inner part, hence taking the form of a disc over the end of the cylinder with a hole at the centre of the disc. The central hole can receive the inlet pipe and also may receive pipework for supply of fluid to fluid inlets.

The outlet may comprise an opening at an outlet end of the drum, for example the outlet end of the drum may be fully open. This enables the material within the drum to exit the drum once it passes out of the final turn of the screw blade. The outlet may include a hopper or similar to receive material that exits the drum and guide it to the next stage of processing.

Since the material in the drum is conveyed via the screw blade then the geometry of a helical screw blade means that if the blade simply terminates without any modification to the form of the final turn(s) of the screw blade then the material will not flow continually out of drum but instead the flow rate will fluctuate. This uneven flow may not be a problem in some circumstances since there may be a possibility to use a hopper or the like as a buffer to gather the material and ensure a continuous flow can be passed to the next stage of processing. However, in some cases it is required to provide a more even flow rate from the outlet of the drum.

In order to provide a more even flow rate from the outlet of the drum then the drum and/or the screw blade may be provided with outlet features during the final turn(s) of the screw blade. The screw blade could be reduced in size toward the outlet end in order to allow for flow of material to spill over the blade and hence exit the drum more evenly. However for materials that are not homogeneous and, for example, include liquid matter as well as solid particles such as bone then this can result in the liquid matter exiting the drum evenly whilst the solid particles, which will settle toward the lower part of the drum and hence not spill over the blade, will still exit at an uneven rate.

An alternative approach is to include holes in the wall of the drum and/or in the surface of the screw blade during the final turn of the screw blade in order to reduce the fluctuations in the flow rate. Holes in the drum wall might require a complicated arrangement to catch the flow from the outlet, but could be beneficial for a relatively non-viscous and homogeneous material. It is also possible to use holes in the wall of the drum to separate liquid and smaller particles, with larger particles exiting the drum from the end of the drum. In this way the rotating drum can be used as a separator.

In one example, holes are provided with openings through the final turn of the screw blade in order to provide for fluid communication between the volume formed between the final and the penultimate turns of the blade and the outlet end of the rotating drum. The holes may be located at the outer perimeter of the screw blade close to the wall of drum and/or at spaced apart locations across the width of the screw blade. These holes may for example be placed in spaced apart locations covering a similar extent of the screw blades to the extent of the mixing vanes. The use of holes in the screw blade can even out the flow rate whilst also ensuring that there is even flow for all parts of the material even if there is a non-homogeneous mixture of, for example, liquid and solid matter. This is since the holes toward the outside of the screw blade, i.e. closest to the wall of the drum, will allow for particles that have settled under gravity to pass through, as well as allowing smaller particles and liquid to pass through. Where the rotating drum is intended for use with materials including solid particles then the size of the holes should be set based on the size of the particles so as to avoid unwanted clogging of the holes.

The holes may be of adjustable size, for example using sliding plates or interchangeable plates. This can allow for adaptation of the rotating drum for differing volumes of material, for differing sizes of solid particles and for differing characteristics of the mixed material, such as solid/liquid ratio, viscosity and so on.

The total area of holes should preferably be sufficient to allow for all of the material within the chamber formed between the final and penultimate turns of the screw to flow out toward the outlet end of the drum through the final turn of the screw blade during one turn of the drum. This would allow for an even flow rate of material out of the outlet from the drum. For typical applications this can be achieved by a total area of holes beneath the expected level of material in the drum that is in the range of 40-200 cm², which can be roughly equated to 180-850 cm² of holes spaced about the circumference of the final turn of the screw blade, assuming that the final turn is open for 90° of the perimeter of the drum, and thus that the holes are spread over 270° of the perimeter. This total size for the holes may be in the context of a drum with diameter in the range 1 to 5 m and overall flow rates in the range of 1000 to 6000 litres per revolution of the drum, i.e. a volume of material of 1000 to 6000 litres held between each pair of turns of the screw blade.

It should be understood that the feature of a horizontal extent of the length of the drum and the axis of rotation of the drum is in order that the material within the drum will gather at a lower part of the drum under the action of gravity in order to thereby enable the action of the screw blade to convey the material along the length of the drum whilst also mixing it in conjunction with the mixing devices. It is not necessary that the length of the drum and the axis of rotation of the drum be completely horizontal. Thus, the drum could be set at an incline in order to also convey the material within the drum vertically upward or downward. In this way the rotating drum apparatus can be used in a similar manner to an Archimedes screw and convey material vertically as well as mixing it. In the case where the inlet of the drum is higher than the outlet of the drum then the weight of the material in the drum may be used to aid rotation of the drum. This might advantageously allow the load on a motor or other drive device for rotation of the drum to be reduced.

The rotating drum apparatus may include a drive device for propelling rotation of the drum, for example a motor attached through suitable gearing to the drum. The rotating drum apparatus may include supports for holding the drum and permitting rotation of the drum, for example supports incorporating bearings. The rotating drum might be held by roller bearings supporting its outer surface, or alternatively the rotating drum might be held via a shaft reported on journal bearings or the like.

The main body of the rotating drum can advantageously be a cylindrical shape, although it will be appreciated that other tubular shapes might be used. An outer perimeter that is circular is generally straightforward to manufacture and could easily be supported for rotation, for example by roller bearings supporting the outer surface of the drum itself. A circular drum also reduces turbulence within the drum itself during rotation, and this can be an advantage for certain types of process. Alternatively, a noncircular drum for example a hexagonal or octagonal prism could be used. A non-circular drum may provide advantages in terms of mixing when a greater degree of turbulence is required.

In one example the drum is arranged to provide a processing capacity of 5 m³ per hour or above, for example about 7 m³ per hour, or in other situations about 30 m³ per hour, or above.

The diameter of the drum may be at least 2 m, for example 2.5 m to 3.5 m. The rotating drum may be arranged so that time taken for the raw material to pass along the extent of the drum is at least 15 minutes, or at least 20 minutes, for example the time taken may be about an hour or more. This allows for sufficient time for reactions to occur and/or for reagents to contact with all of the raw material.

The length of the drum between the inlet and the outlet may for example be 3 m or more, for example 5.5 m or above 10 m.

The pitch of the drum may be chosen to provide a desired number of chambers within the drum, and to provide a desired residence time of the material in the drum (in combination with the length of the drum and the speed of rotation of the drum). The ratio of the pitch to the length of the drum may for example be approximately 1:20, approximately 1:30 or approximately 1:40.

The inlet and the outlet may be at the ends of the drum. The diameter of the drum, the length of the drum and the speed of rotation of the drum may be set so as to provide a processing capacity as set forth above.

In one example the length of the drum is 11.75 m, the diameter of the drum is 3.5 m, the screw blade extends by 1.25 m into the centre of the drum from the outer wall, the pitch of the screw blade is 0.375 m, and the mixing vanes have a height of 0.5 m. In this example there are five mixing vanes for each turn of the screw blade and there may be five fluid inlets spaced apart along the extent of the trailing edge of the mixing vane. This drum can be operated to process about 30 m³ of material an hour in the form of 15 tonnes of raw material and 15 tonnes of water, with the travel time from the inlet to the outlet being about 1 hour.

Optionally, the acid solution used in demineralisation of the mineralised tissue remnants comprises hydrochloric acid. The acid solution may alternatively or additionally comprise other strong acids such as sulphuric acid (H₂SO₄), nitric acid (HNO₃) and phosphoric acid (H₃PO₄). The stronger the acid the more effective it will be at demineralising the mineralised tissue remnants. It has been found that hydrochloric acid (HCl) has an advantageous dissociation constant and is more economically viable than other acids. The use of HCl is also more practically viable and has lower risks and associated costs relating to the health and safety of using strong acids.

Optionally, the acid solution comprises acid at a concentration of between 1 and 5 wt %. Optionally, the concentration of acid is between 1 and 4 wt %, or between 2 and 3 weight percent. Preferably the concentration of acid is approximately 3 wt %.

The concentration of acid may depend on the composition of the mineralised tissue remnants and hence the type of organic feedstock that is used. The concentration of acid may also depend on the type of acid that is used. The concentration of acid should be high enough so that the rate of the demineralisation reaction is not unduly inhibited by a lack of an acidic reactant, but should also not be so high that unreacted acid is wasted. Both of these factors increase the economic efficiency of the demineralisation process.

Optionally, the weight ratio of mineralised tissue remnant to acid solution is between 1:2 and 1:8.

Optionally, the weight ratio of mineralised tissue remnant to acid solution is between 1:3 and 1:5. Optionally, the weight ratio of mineralised tissue remnant to acid solution is 1:5. Thus, there is a greater weight of acid solution compared to the weight of mineralised tissue remnant. This ensures that the acid solution covers all of the mineralised tissue remnants during the demineralisation so that the rate of reaction is not limited by a lack of contact between the acid and the mineralised tissue remnants. The weight proportion of acid solution is limited for spatial and economic efficiency and to limit the amount of acid solution wasted. The lower the proportion of acid solution used the more mineralised tissue remnant can be processed within the same volume of container.

Optionally, the demineralisation of the mineralised tissue remnants is carried out for between 30 to 180 minutes.

Optionally, the demineralisation of the mineralised tissue remnants is carried out for between 60 to 120 minutes. Optionally, the demineralisation of the mineralised tissue remnants is carried out for approximately 60 minutes. When the demineralisation process is carried out in a continuous flow process, the length of time for which demineralisation is carried out is dependent on the residence time of the mineralised tissue remnant within the reaction vessel. The residence time of the mineralised tissue remnants within the reaction vessel can be controlled by varying the length of the path between the inlet and outlet of the reaction vessel which the mineralised tissue remnants must travel. The speed at which the mineralised tissue remnants travel through the reaction vessel can also determine the duration of the demineralisation process. In the case of a rotating drum reaction vessel, the speed at which the mineralised tissue remnants travel between the inlet and the outlet of the drum (i.e. along the axial length of the drum) depends on the speed of rotation of the drum. The length of time of the demineralisation process should be sufficient for substantially all of the mineral portion of the mineralised tissue remnants to be solubilised, but not so long that energy is wasted by maintaining completely demineralised tissue remnants in the demineralisation process unnecessarily.

The mineralised tissues to be mixed with acid in the drum may comprise a proteinaceous portion. The demineralisation of the mineralised tissues may be carried out until the proteinaceous portion of the mineralised tissues is partially or completely separated from the mineral portion of the mineralised tissues.

The mineralised tissues may be derived from an organic feedstock derived from or comprising meat offcuts, including for example fish offcuts (for example salmon or cod), or poultry offcuts, or other animal derivatives. In particular, the organic feedstock may be derived from waste produced by the food industry, or may be a secondary product of another industrial process. The organic feedstock may comprise “industrial fish”, i.e. raw materials used for fish meal production. The processing may therefore aid in utilisation of discarded products and minimisation of waste.

Depending on the organic feedstock, the mineralised tissues present in the organic feedstock may comprise one of more of: bones, fish scales, tendons, cartilage, tooth enamel, dentin, and hooves. Preferably, the mineralised tissue comprises bones. The organic feedstock may comprise flesh and bones from aquatic animal origin (for example, marine animal origin) or terrestrial animal origin.

The mineralised tissues within the organic feedstock may comprise a proteinaceous portion and a mineral portion. In the case that the mineralised tissue is bone, the mineral portion of the mineralised tissues may comprise calcium hydroxyapatite. In the case that the mineralised tissue is bone, the proteinaceous portion may comprise ossein (comprising predominantly collagen).

When the mineralised tissues comprise a proteinaceous portion, the demineralisation of the mineralised tissues will result in demineralised tissues comprising the proteinaceous portion. These demineralised tissues may then be further treated, for example the demineralised tissues may undergo a further enzymatic hydrolysis treatment (for example, using a protease). Enzymatic hydrolysis of the demineralised tissues may then result in at least part of the proteinaceous portion of the demineralised tissues being hydrolysed.

Optionally, the demineralisation of the mineralised tissue remnants is carried out at a temperature of between 30 and 70 degrees centigrade. Optionally, the demineralisation of the mineralised tissue remnants is carried out at a temperature of between 40 and 55 degrees centigrade. Optionally, the demineralisation of the mineralised tissue remnants is carried out at a temperature of approximately 40 degrees. Thus the temperature at which the demineralisation is carried out is advantageously elevated so as to provide energy to the reactants for an increased reaction rate without causing damage to the reactants or products and without waste of energy or other resources by raising the temperature unnecessarily high.

Structural features of the rotating drum apparatus have been described above with particular reference to their functionality when the drum is used in a method of demineralising mineralised tissue remnants. The same structures also may be used for the same or different functionality where the rotating drum apparatus is used as the reaction vessel for carrying out enzymatic hydrolysis (for example, in any of the first, second, third or fourth aspects of the invention). For example, the fluid inlets of the rotating drum may be used for differing purposes, as discussed in more detail below.

Where the rotating drum apparatus is used as the reaction vessel for carrying out enzymatic hydrolysis, the fluid inlets may allow for the addition of reagents to change the characteristics of the material in the drum. The reagent may be an acid, a base, water, an organic solvent, or a solution such as water containing salt or buffer for example.

The fluid inlets may be arranged for the introduction of gases into the material in the drum via the fluid inlets, for example the introduction of inert gases in order to remove oxygen and other reactive gases from the material being processed.

The fluids introduced by the fluid inlets may be at an elevated or lowered temperature compared to the temperature of the materials within the drum. In this way the addition of fluids by the fluid inlets can both prompt mixing of the materials within the drum and also adjust the temperature thereof. For example, hot water could be added to increase the temperature to prompt an enzymatic reaction in later parts of the drum apparatus or in a subsequent processing area, or a hot gas could be bubbled through the material for the same purpose. Alternatively, cold liquid (e.g. water) or a cold gas could be introduced to reduce the temperature, for example to cause an enzymatic reaction to cease. Similarly, hot liquid or gas could be introduced to increase the temperature, for example to inactivate/denature the enzymes to cause an enzymatic reaction to cease.

Optionally, steam maybe introduced by the fluid inlets through fluid inlets close to the outlet of the drum, to cause enzymatic reactions to cease (by inactivating the enzymes).

It is noted that the use of the rotating drum apparatus is suitable for enzymatic processing as it can avoid the formation of emulsions. Avoiding or reducing the formation of emulsions is an important consideration in enzymatic processing systems (for example, hydrolysis of protein/lipid mixtures). Emulsions block enzymatic access to parts of the feedstock trapped in emulsions and thus reduce the efficiency of enzymatic processing. Furthermore, the problem with emulsions extends to the separation stage. In emulsions, lipids may be tightly associated with water-soluble components such as peptide material which mechanical separators are unable to separate. Thus the result can be poor separation with, for example, lipid in the protein phase and/or protein in the lipid phase. Emulsions can be taken out by filtration at a later stage, but the emulsified components still cannot be recovered and combined with the non-emulsified fractions. That is, without specific equipment it is not possible to separate water-soluble components from the emulsion to recombine them with the non-emulsified water-soluble fraction, and nor is it possible to separate lipids and lipid-soluble components from the emulsion to recombine them with the non-emulsified lipid and lipid-soluble fraction.

Certain embodiments of the invention will now be described by way of example only and with reference to the accompanying drawings in which:

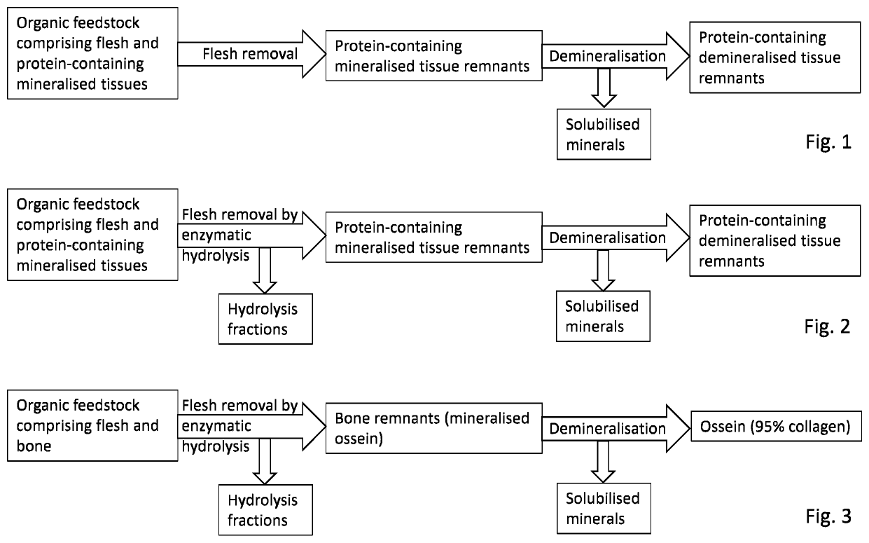

FIG. 1 illustrates a method of processing an organic feedstock comprising flesh and protein-containing mineralised tissues;

FIG. 2 illustrates another method of processing an organic feedstock comprising flesh and protein-containing mineralised tissues;

FIG. 3 illustrates a method of processing an organic feedstock comprising flesh and bone;

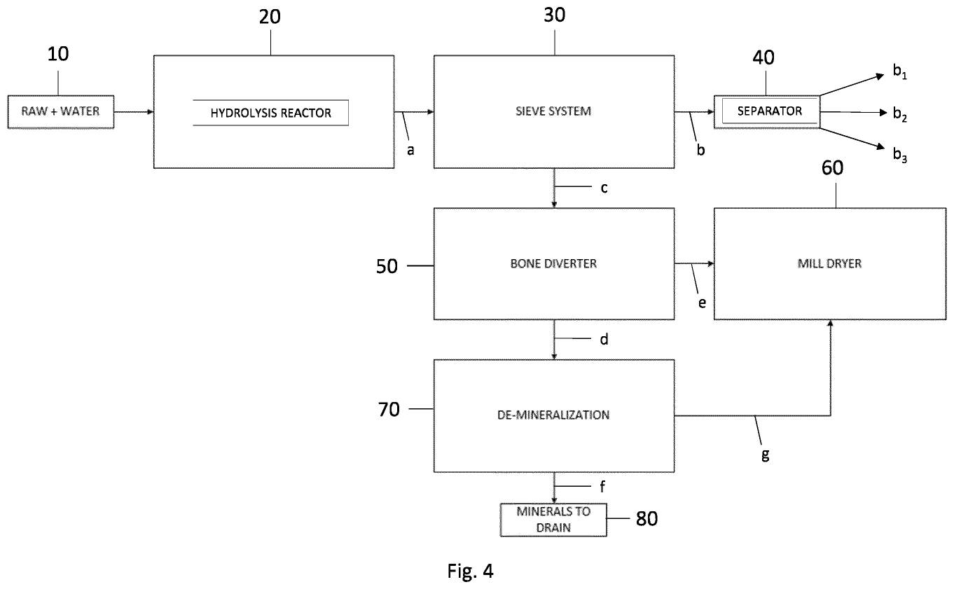

FIG. 4 is a schematic illustrating a first processing plant;

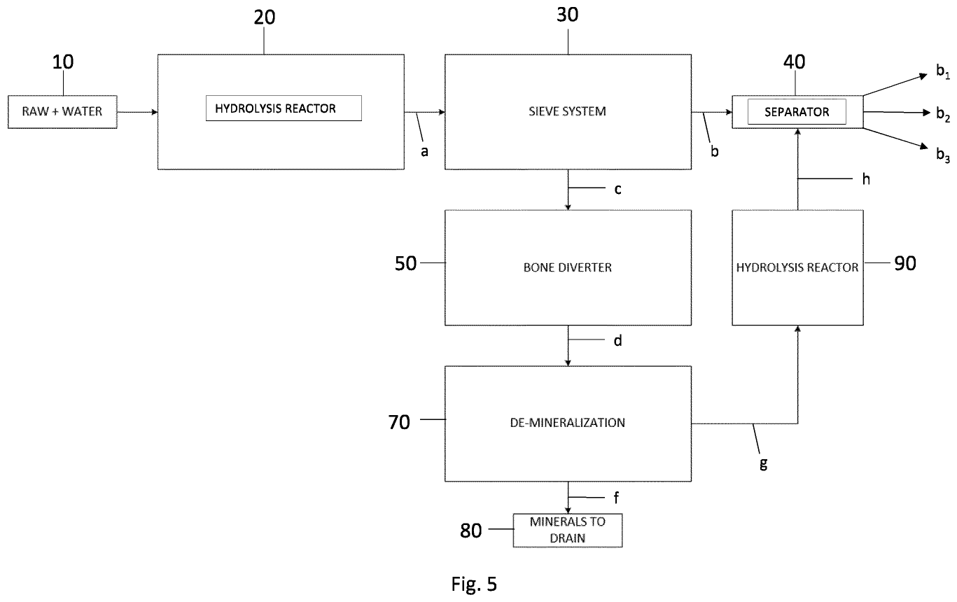

FIG. 5 is a schematic illustrating a second processing plant;

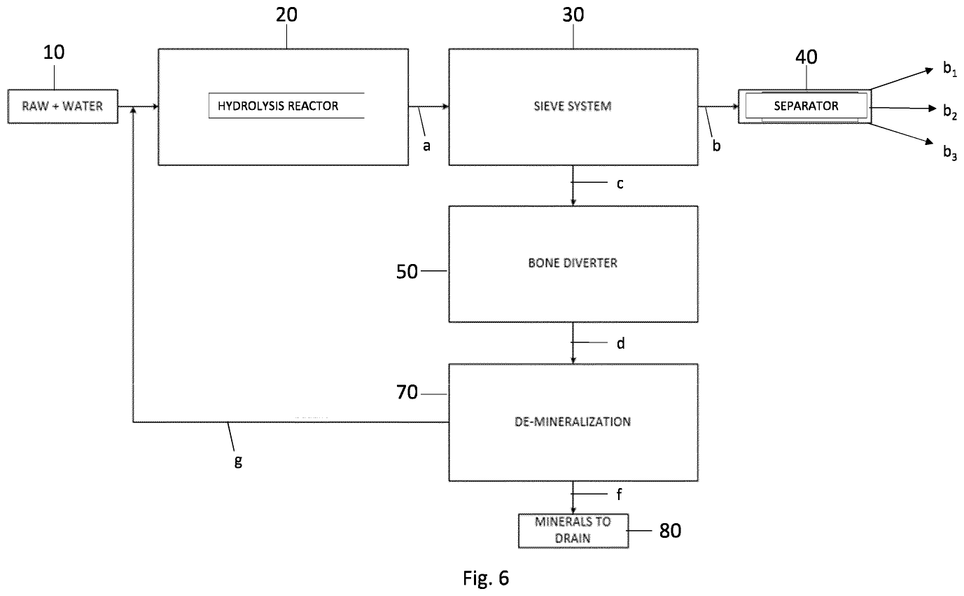

FIG. 6 is a schematic illustrating a third processing plant;

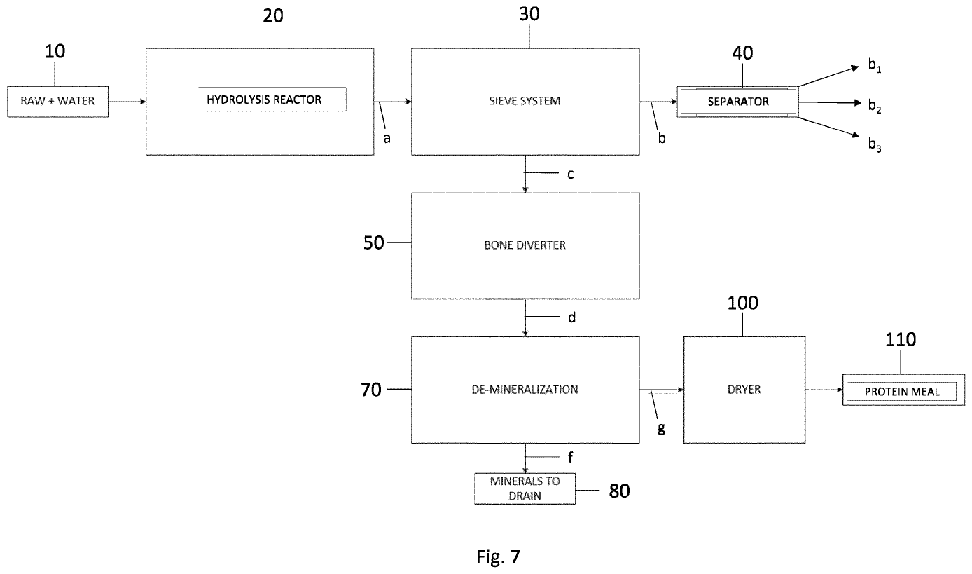

FIG. 7 is a schematic illustrating a fourth processing plant;

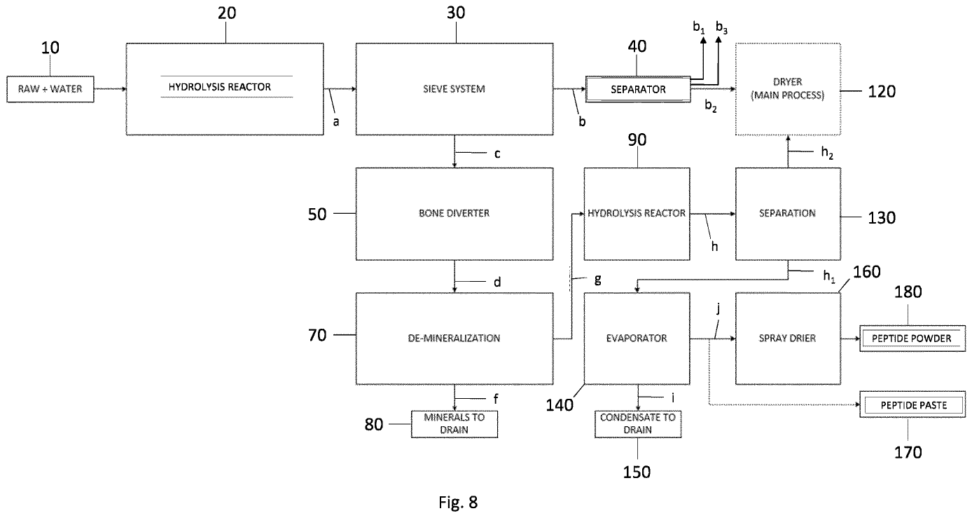

FIG. 8 is a schematic illustrating a fifth processing plant;

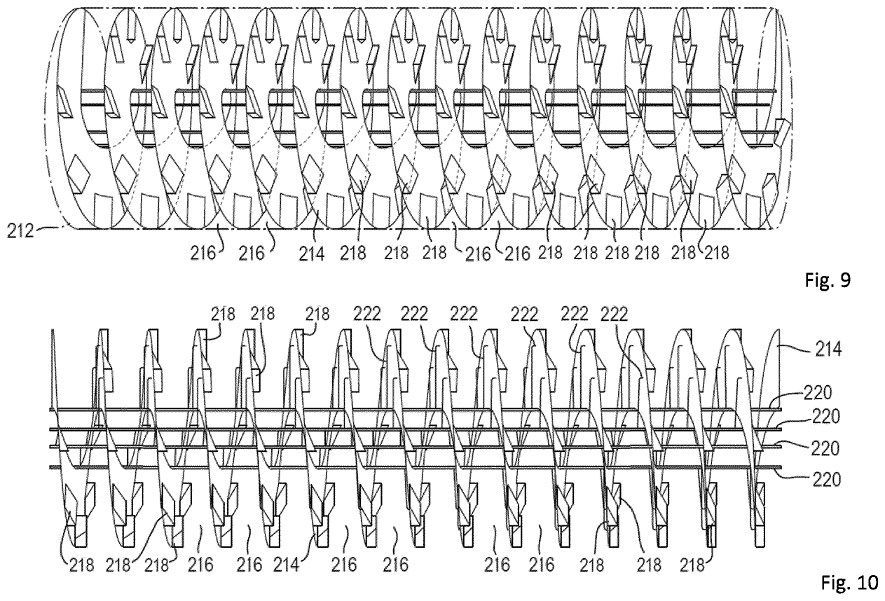

FIG. 9 illustrates a drum for rotation to mix material within the drum and convey the mixture along the length of the drum;

FIG. 10 shows a helical blade with mixing devices as used in the drum of FIG. 9;

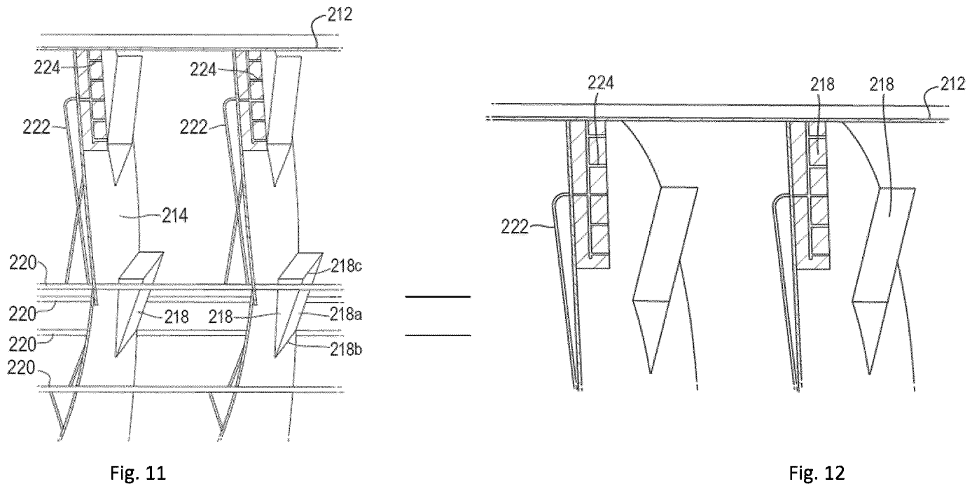

FIG. 11 is a close up view of a part of the helical blade of FIG. 10;

FIG. 12 is a further close up of a part of FIG. 11;

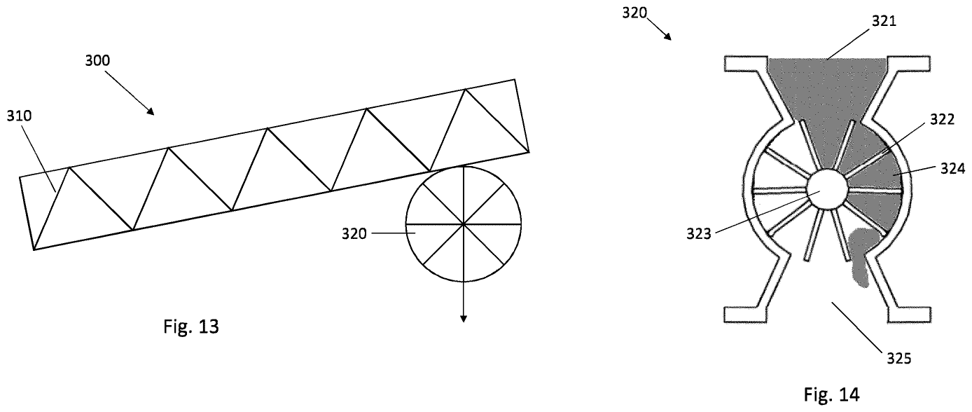

FIG. 13 shows a possible configuration for a diverter for separating a flow of mineralised tissue remnants; and

FIG. 14 shows a rotary dosing valve forming part of the diverter of FIG. 13.

FIG. 1 shows a method of processing an organic feedstock. Here, the organic feedstock comprises flesh and protein-containing mineralised tissues. The protein-containing mineralised tissues typically comprise a proteinaceous organic matrix (i.e. an organic matrix predominantly formed of protein) and minerals bound into the proteinaceous organic matrix. Thus, the protein-containing mineralised tissues predominantly consist of protein and minerals.

The first step in the process comprises the removal of flesh. This leaves behind protein-containing mineralised tissue remnants. Removal of the flesh allows for the protein-containing mineralised tissue remnants to be more easily processed subsequently.

The next step comprises the demineralisation of the protein-containing mineralised tissue remnants, resulting in the production of solubilised minerals which can be readily separated from the demineralised protein-containing tissue remnants.

The demineralised protein-containing tissue remnants can then be treated further to obtain desired end products (as discussed in detail below).

In the protein-containing mineralised tissues, the minerals form a barrier which prevents (or at least reduces the rate of) chemical reactions involving the proteinaceous organic matrix. The removal of the minerals therefore means that the protein within the demineralised protein-containing tissue remnants can more readily react, compared to the protein within the protein-containing mineralised tissues.

FIG. 2 shows a similar method to that discussed in FIG. 1 (with all of the above comments in respect of the method of FIG. 1 applying also to the method of FIG. 2), the only difference being that in the method of FIG. 2, the means of flesh removal is specified as being enzymatic hydrolysis. Since flesh is predominantly proteinaceous, the enzyme used in the enzymatic hydrolysis process is a protease. It is noted that the enzymatic hydrolysis is predominantly a reaction with the flesh (i.e. hydrolysis of the flesh); the protein in the protein-containing mineralised tissues is shielded from the enzymatic hydrolysis process by the minerals bound within the proteinaceous matrix, and so is not readily hydrolysed.

FIG. 3 shows a similar method to that discussed in FIG. 2 (with all of the above comments in respect of the method of FIGS. 1 and 2 applying also to the method of FIG. 3), except that in FIG. 3, it is specified that the organic feedstock comprises flesh and bone. In bone, the organic proteinaceous matrix makes up about 30 to 35% of the bone mass, and is referred to as ossein. Ossein comprises approximately 95% collagen (in the form of flexible elastic fibres), which is a protein. The mineral bound into the ossein matrix is predominantly calcium phosphate, in a chemical arrangement known as calcium hydroxyapatite.

The first step in the process of FIG. 3 comprises the removal of flesh by enzymatic hydrolysis (for example using a protease). This leaves behind protein-containing mineralised tissue remnants, i.e. mineralised ossein. Removal of the flesh allows for the mineralised ossein to be more easily processed subsequently.

The next step comprises the demineralisation of the mineralised ossein, resulting in the production of solubilised minerals which can be readily separated from the ossein.

The ossein (i.e. the demineralised protein-containing tissue remnants) can then be treated further to obtain desired end products (as discussed in detail below).

In all three of the foregoing methods, some or all of the demineralised protein-containing tissue remnants can be further processed by, for example:
A further step of enzymatic hydrolysis (for example comprising the use of a protease) in order to hydrolyse the demineralised protein-containing tissue remnants;
Drying to form a protein meal.

Both processes are discussed in further detail below.

In all three of the foregoing methods, a portion of the mineralised protein-containing tissue remnants can be further processed by, for example:
Drying (optionally in a mill dryer).

In any of the above three methods incorporating one or more steps of enzymatic hydrolysis (optionally using a protease) the hydrolysis fractions may include the following fractions:
an aqueous fraction comprising water with dissolved protein, polypeptides and amino acids (the aqueous fraction may be referred to as a hydrolysate);
a sediment fraction comprising for example insoluble proteins and other small solids (the sediment fraction may typically comprise 30-40% dry matter); and
an oil fraction.

The hydrolysis fractions may be separated into the three component fractions, and each of the aqueous fraction, sediment fraction and oil fraction may be further treated once separated. The aqueous fraction may be dried. The sediment fraction may be dried. The aqueous fraction and sediment fraction need not be fully dried, but may be partially dried. The aqueous fraction and sediment fraction may be dried to the extent that they are in a form which is stable according to its water activity. For example, the aqueous fraction may be partially dried to form a paste, usually with above 60% dry matter content.

FIG. 4 is a schematic illustrating a first processing plant. As seen in FIG. 4, raw material (i.e. organic feedstock comprising mineralised tissues) is mixed with water in a first stage 10, and then the mixture is transferred to a first reaction vessel 20 in which enzymatic hydrolysis of the organic feedstock is carried out.

The enzymatic hydrolysis typically involves heating the organic feedstock with water. The process is carried out at a temperature of approximately 55° C. and at a pH of approximately pH 6. Enzymes, e.g. protease, are added and the solution is mixed for between 40 to 120 minutes.

The output from the hydrolysis of the organic feedstock (labelled “a” in FIG. 4) comprises:
an aqueous fraction comprising water with dissolved protein, polypeptides and amino acids (and also any molecule dissolved in the water);
a sediment fraction comprising insoluble proteins and other small solids;
an oil fraction; and
a mineralised tissue remnant fraction comprising larger solids.

The first two of these are together termed “the slurry fraction”; so the output from the first reaction vessel 20 comprises a slurry fraction, an oil fraction, and a mineralised tissue remnant fraction.

The output of the first reaction vessel 20 is received by a first separator 30 configured to separate the mineralised tissue remnants from the slurry fraction and oil fraction. The first separator 30 comprises a vibrating sieve system. As noted above, the slurry fraction includes a sediment fraction comprising insoluble proteins and other small solids-here, the small solids (and indeed insoluble proteins) are smaller than then holes in the sieve, such that they pass through the sieve. The aqueous fraction also passes through the sieve, as does the oil fraction.

The slurry fraction and oil fraction which pass through the first separator 30 (labelled “b” in FIG. 4) are received by a hydrolysis fractions separation vessel 40. In this example, the hydrolysis fractions separation vessel 40 is a three-phase centrifugal decanter, but other configurations are possible (for example, a combination between a two phase decanter and one or two separators). Whatever the structural configuration, functionally the hydrolysis fractions separation vessel 40 is configured to separate the slurry fraction and oil fraction into:
b₁: an aqueous fraction comprising water with dissolved protein, polypeptides and amino acids;
b₂: a sediment fraction comprising insoluble proteins and other small solids; and
b₃: an oil fraction.

The mineralised tissue remnant fraction retained by the first separator 30 (labelled “c” on FIG. 4) i.e. comprising solids too large to pass through the sieve, is received by a diverter 50 wherein a first portion of the mineralised tissue remnant fraction (labelled “e” on FIG. 4) is diverted to a dryer 60, and a second portion of the mineralised tissue remnant fraction (labelled “d” on FIG. 4) is diverted to a second reaction vessel 70.

In this example, the first portion of the mineralised tissue remnant fraction diverted to the dryer 60 comprises around 60% by weight of the solid fraction. However, this is by no means limiting—the relative amounts of fractions “e” and “d” may be chosen according to the particular biorefining process and desired end products; there is no generally applicable ratio.

The dryer 60 is a mill dryer which simultaneously grinds and dries the solid fraction.

The second portion of the mineralised tissue remnant fraction comprises the remaining portion of the mineralised tissue remnant fraction after the first portion is removed. That is, in this specific non-limiting example, the second portion comprises around 40% by weight of the mineralised tissue remnant fraction. The second portion is received by the second reaction vessel 70 and undergoes a demineralisation process.

The demineralisation process carried out in the second reaction vessel 70 typically involves heating the mineralised tissue remnants with an HCl acid solution at a concentration of approximately 3 wt %. The process is carried out at a temperature of approximately 40° C. The weight ratio of mineralised tissue remnants to acid solution is approximately 1:5. The mineralised tissue remnants and the acid solution are mixed for approximately 60 minutes before returning to room temperature, i.e. the demineralisation process is carried out for approximately 60 minutes.

The output of the second reaction vessel 70 comprises a liquid fraction comprising solubilised minerals (labelled “f” on FIG. 4) and a demineralised tissue remnant fraction (labelled “g” on FIG. 4) comprising insoluble proteinaceous material, such as ossein. The liquid fraction of the output of the second reaction vessel 70 (labelled “f” on FIG. 4) is, in this example, output to a drain 80 for collection (the solubilised minerals can then be collected and can be advantageously utilised in further processes or products) or for disposal. The solid fraction of the output of the second reaction vessel 70 (labelled “g” on FIG. 4) is directed to the dryer 60.

FIG. 5 shows another processing plant. In FIG. 5, the components a, b, c, d, f and g are the same as those described in reference to FIG. 4, and the processes carried out by the first reaction vessel 20, first separator 30, hydrolysis fractions separation vessel 40, and second reaction vessel 70 are as described in reference to FIG. 4.

In the processing plant of FIG. 5, raw material (i.e. the organic feedstock comprising mineralised tissues) is mixed with water in a first stage 10, and the mixture is transferred to a first reaction vessel 20 in which enzymatic hydrolysis of the organic feedstock is carried out as described in relation to FIG. 4.

The hydrolysis of the organic feedstock results in the formation of the following (labelled “a” on FIG. 5): an aqueous fraction comprising water with dissolved protein, polypeptides and amino acids; a sediment fraction comprising insoluble proteins and other small solids; an oil fraction; and a mineralised tissue remnant fraction comprising larger solids. The output of the first reaction vessel 20 is received by a first separator 30 comprising a vibrating sieve.

The slurry fraction and oil fraction (labelled “b” on FIG. 5) which pass through the first separator 30 are received by a hydrolysis fractions separation vessel 40 where they are separated into: an aqueous fraction comprising water with dissolved protein, polypeptides and amino acids (labelled “b₁”); a sediment fraction comprising insoluble proteins and other small solids (labelled “b₂”); and an oil fraction (labelled “b₃”).

The mineralised tissue remnant fraction retained by the first separator 30 (labelled “c” on FIG. 5) i.e. comprising solids too large to pass through the sieve, is received by a diverter 50 and directed to the second reaction vessel 70 where the demineralisation process is carried out as described in relation to FIG. 4. In this embodiment the diverter does not separate the mineralised tissue remnant fraction into separate portions; it simply passes all of the mineralised tissue remnant fraction (labelled “d” on FIG. 5) on to second reaction vessel 70. The diverter is present to allow greater flexibility of the system.

The output of the second reaction vessel 70 comprises a liquid fraction (labelled “f” on FIG. 5) comprising solubilised minerals and a demineralised tissue remnant fraction (labelled “g” on FIG. 5) comprising insoluble proteinaceous material, such as ossein. The liquid fraction (f) of the output of the second reaction vessel is, in this example, output to a drain 80 for collection (the solubilised minerals can then be collected and can be advantageously utilised in further processes or products) or for disposal.

The demineralised tissue remnants fraction output from the second reaction vessel 70 is directed to a third reaction vessel 90 in which enzymatic hydrolysis of the demineralised tissue remnants (g) output from the second reaction vessel is carried out.

The enzymatic hydrolysis carried out in the third reaction vessel 90 may be carried out using similar parameters to those described in relation to the enzymatic hydrolysis carried out within the first reaction vessel 20. Alternatively, the exact parameters chosen for the second hydrolysis process may be tailored to the reactants involved. That is the parameters may be different for the second enzymatic hydrolysis process on account of the differing input material (e.g. in the first reaction vessel 20, the feedstock may comprise flesh and bones, whereas in the third reaction vessel 90, the feedstock comprises demineralised tissue remnants).

The output of the third reaction vessel 90 (labelled “h” on FIG. 5) comprises an aqueous fraction comprising water with dissolved protein, polypeptides and amino acids; and a sediment fraction comprising insoluble proteins and other small solids. The output of the third reaction vessel 90 (labelled “h”) is received by the hydrolysis fractions separation vessel 40 where it is combined with the fraction labelled “b” described above, and is separated as discussed above.

FIG. 6 shows another processing plant. In FIG. 6, the components a, b, c, d, f and g are the same as those described in reference to FIG. 4, and the processes carried out by the first reaction vessel 20, first separator 30, hydrolysis fractions separation vessel 40, and second reaction vessel 70 are as described with reference to FIG. 4.

In the processing plant of FIG. 6, raw material (i.e. the organic feedstock comprising mineralised tissues) is mixed with water in a first stage 10, and the mixture is transferred to a first reaction vessel 20 in which enzymatic hydrolysis of the organic feedstock is carried out as described in relation to FIG. 4.

The hydrolysis of the organic feedstock results in the formation of the following (labelled “a” on FIG. 6): an aqueous fraction comprising water with dissolved protein, polypeptides and amino acids; a sediment fraction comprising insoluble proteins and other small solids; an oil fraction; and a mineralised tissue remnant fraction comprising larger solids.

The output of the first reaction vessel is received by a first separator 30 comprising a vibrating sieve configured to separate the mineralised tissue remnant fraction out from the remainder of the material output from the first reaction vessel 20.

The slurry fraction and oil fraction (labelled “b” on FIG. 6) which pass through the first separator 30 are received by a hydrolysis fractions separation vessel 40 where they are separated into: an aqueous fraction comprising water with dissolved protein, polypeptides and amino acids (labelled “b₁”); a sediment fraction comprising insoluble proteins and other small solids (labelled “b₂”); and an oil fraction (labelled “b₃”).

The mineralised tissue remnant fraction retained by the first separator 30 (labelled “c” on FIG. 6) i.e. solids too large to pass through the sieve, is received by a diverter 50 and directed to the second reaction vessel 70 where the demineralisation process is carried out as described in relation to FIG. 4. In this embodiment the diverter does not separate the mineralised tissue remnant fraction into a plurality of portions, but is present for flexibility of the system.

The output of the second reaction vessel 70 comprises a liquid fraction (labelled “f” on FIG. 6) comprising solubilised minerals and a demineralised tissue remnant fraction (labelled “g” on FIG. 6) comprising insoluble proteinaceous material. The liquid fraction (f) of the output of the second reaction vessel is sent to a drain 80 for collection (the solubilised minerals can then be collected and can be advantageously utilised in further processes or products) or for disposal.

The demineralised tissue remnant fraction (g) output from the second reaction vessel 70 is directed to and received by the first reaction vessel 20. The demineralised tissue remnants are then subjected to a further hydrolysis process in order to obtain further proteins, polypeptides and amino acids. In this way the demineralised tissue remnants are recycled to the first reaction vessel 20 and incorporated with further raw material (organic feedstock comprising mineralised tissues and water). The hydrolysis of the demineralised tissue remnants within the first reaction vessel 20 may be more complete than the hydrolysis of the original mineralised tissue remnants in the first pass through the reaction vessel 20. Due to the recycling and re-hydrolysis of the material a higher yield of desirable end products can be achieved.

FIG. 7 shows another processing plant. In FIG. 7, the components a, b, c, d, f and g are the same as those described in reference to FIG. 4, and the processes carried out by the first reaction vessel 20, first separator 30, hydrolysis fractions separation vessel 40, and second reaction vessel 70 are as described with reference to FIG. 4.

In the processing plant of FIG. 7, raw material (i.e. organic feedstock comprising mineralised tissues) is mixed with water in a first stage 10, and then the mixture is transferred to a first reaction vessel 20 in which enzymatic hydrolysis of the organic feedstock is carried out as described in relation to FIG. 4.

The hydrolysis of the organic feedstock within the first reaction vessel 20 results in the formation of the following (labelled “a” on FIG. 7): an aqueous fraction comprising water with dissolved protein, polypeptides and amino acids; a sediment fraction comprising insoluble proteins and other small solids; an oil fraction; and a mineralised tissue remnant fraction comprising larger solids.

The output of the first reaction vessel is received by a first separator 30 comprising a vibrating sieve configured to separate the mineralised tissue remnants fraction out from the remainder of the material output from the first reaction vessel.

The slurry fraction and oil fraction (labelled “b” on FIG. 7) which pass through the first separator 30 are received by a hydrolysis fractions separation vessel 40 where they are separated into: an aqueous fraction comprising water with dissolved protein, polypeptides and amino acids (labelled “b₁”); a sediment fraction comprising insoluble proteins and other small solids (labelled “b₂”); and an oil fraction (labelled “b₃”).

The mineralised tissue remnants fraction retained by the first separator 30 (labelled “c” on FIG. 7) i.e. comprising solids too large to pass through the sieve, is received by a diverter 50 and directed to the second reaction vessel 70 where the demineralisation process is carried out as described in relation to FIG. 4. In this embodiment the diverter does not separate the mineralised tissue remnants fraction into a plurality of portions, but is present for flexibility of the system.

The output of the second reaction vessel 70 comprises a liquid fraction (labelled “f”) comprising solubilised minerals and a demineralised tissue remnant fraction (labelled “g”) comprising insoluble proteinaceous material. The liquid fraction (f) of the output of the second reaction vessel is directed to a drain 80 for collection (the solubilised minerals can then be collected and can be advantageously utilised in further processes or products) or for disposal.

The demineralised tissue remnant fraction (g) output from the second reaction vessel 70 is directed to and received by a dryer 100. The demineralised tissue remnant fraction of the output of the second reaction vessel 70 is dried in order to obtain a protein meal 110, such as collagen meal.

FIG. 8 shows another processing plant. In FIG. 8, the components a, b, c, d, f, g and h are the same as those described in reference to FIG. 4 and FIG. 5, and the processes carried out by the first reaction vessel 20, first separator 30, hydrolysis fractions separation vessel 40, and second reaction vessel 70 are as described with reference to FIG. 4.

In the processing plant of FIG. 8, raw material (i.e. the organic feedstock comprising mineralised tissues) is mixed with water in a first stage 10, the mixture is transferred to a first reaction vessel 20 in which enzymatic hydrolysis of the organic feedstock is carried out as described in relation to FIG. 4.

The hydrolysis of the organic feedstock within the first reaction vessel 20 results in the formation of the following (labelled “a” on FIG. 8): an aqueous fraction comprising water with dissolved protein, polypeptides and amino acids; a sediment fraction comprising insoluble proteins and other small solids; an oil fraction; and a mineralised tissue remnant fraction comprising larger solids.

The output of the first reaction vessel 20 is received by a first separator 30 comprising a vibrating sieve configured to separate the mineralised tissue remnant fraction out from the remainder of the material output from the first reaction vessel 20.

The slurry fraction and oil fraction (labelled “b” on FIG. 8) which passes through the first separator 30 are received by a hydrolysis fractions separation vessel 40 where they are separated into: an aqueous fraction comprising water with dissolved protein, polypeptides and amino acids (labelled “b₁”); a sediment fraction comprising insoluble proteins and other small solids (labelled “b₂”); and an oil fraction (labelled “b₃”). The sediment fraction comprising insoluble proteins and other small solids (labelled “b₂”) is then directed to a dryer 120 in order to form protein meal comprising insoluble proteins.

The mineralised tissue remnant fraction retained by the first separator 30 (labelled “c” on FIG. 8) i.e. solids too large to pass through the sieve, is received by a diverter 50 and directed to the second reaction vessel 70 where the demineralisation process is carried out as described in relation to FIG. 4. In this embodiment the diverter does not separate the mineralised tissue remnant fraction into a plurality of portions, but is present for flexibility of the system.

The output of the second reaction vessel 70 comprises a liquid fraction (labelled “f”) comprising solubilised minerals and a demineralised tissue remnant fraction (labelled “g”) comprising insoluble proteinaceous material. The liquid fraction (f) of the output of the second reaction vessel 70 is output to a drain 80 for collection (the solubilised minerals can then be collected and can be advantageously utilised in further processes or products) or for disposal.

The demineralised tissue remnant fraction (g) output from the second reaction vessel 70 is directed to and received by a third reaction vessel 90 wherein the demineralised tissue remnants undergo a further enzymatic hydrolysis process. The enzymatic hydrolysis carried out in the third reaction vessel 90 may be carried out using similar parameters to those described in relation to the enzymatic hydrolysis carried out within the first reaction vessel 20. Alternatively, the exact parameters chosen for the second hydrolysis process may be tailored to the reactants involved. That is the parameters may be different for the second enzymatic hydrolysis process on account of the differing input material (e.g. in the first reaction vessel 20, the feedstock may comprise flesh and bones, whereas in the third reaction vessel 90, the feedstock comprises demineralised tissue remnants).

The output of the third reaction vessel 90 (labelled “h”) comprises: an aqueous fraction (labelled “h₁”) comprising water with dissolved protein, polypeptides and amino acids; and a sediment fraction (labelled “h₂”) comprising insoluble proteins and other small solids. The output of the third reaction vessel 90 (labelled “h”) is then received by a second separator 130 where the sediment fraction comprising insoluble proteins and other small solids (h₂) is separated from the aqueous fraction (h₁).

The sediment fraction (labelled “h₂”) comprising insoluble proteins and other small solids is directed to and received by the dryer 120 (along with fraction b₂, described above) where it is dried to form protein meal comprising insoluble proteins.

The aqueous fraction (labelled “h₁”) comprising water with dissolved protein, polypeptides and amino acids is received by an evaporator 140 configured to dry the aqueous fraction. The evaporated gaseous portion is directed to a condenser 150 from which the condensate (labelled “i”) is then drained for collection or disposal. The paste (labelled “j”) resulting from the evaporation process may be collected as a peptide paste 170. As an example the peptide paste is collagen peptide paste. Here, it should be understood that the collagen peptide paste does not comprise collagen (since the collagen was hydrolysed in the second enzymatic hydrolysis process carried out in the third reaction vessel 90), instead, the paste comprises collagen-derivatives, i.e. proteins, polypeptides and amino acids resulting from the hydrolysis of collagen.

A proportion of the peptide paste (from 0% to 100%) may be directed to a spray dryer 160 in order to produce a peptide powder 180. As an example the peptide powder is collagen peptide powder (comprising proteins, polypeptides and amino acids resulting from the hydrolysis of collagen). The proportion of the peptide paste directed to the spray dryer 160 to produce peptide powder is simply dependent on the desired yield of each product.

FIGS. 9 to 12 show a rotatable drum that can be used in a rotating drum apparatus for mixing and conveying raw materials, such as for mixing raw materials/reactants for the enzymatic hydrolysis and demineralisation processes explained above.

As can be seen in FIG. 9, the rotatable drum has a cylindrical shape with an outer wall formed as a cylindrical tube 212. In use, the drum is arranged to have its axis of rotation extending along the horizontal. An inlet for the flow of reactants is positioned on a first end of the drum, and an outlet is positioned on the second end of the drum downstream of the inlet. The reactants are conveyed along the axial length of the drum from the inlet to the outlet. A screw comprising a helical blade 214 is provided within the cylindrical tube 212 with the outer edge of the helical blade 214 being fixed to the inner wall of the cylindrical tube 212. This may be done, for example, by welding. It is beneficial to ensure that a watertight seal is formed between the outer edge of the helical blade 214 and the inner wall of the cylindrical tube 212, since this means that multiple chambers 216 can be formed. A chamber 216 is formed between two adjacent turns of the helical blade 214. The chambers are open to a channel extending axially through the centre of the drum above the height of the helical blade 214 (the helical blade does not extend along the entire radius of the drum).

The helical blade 214 of the drum illustrated in FIGS. 9 and 10 has a constant pitch throughout the length of the drum such that the chambers formed between each turn of the blade maintain a constant volume. In other designs, the pitch of the helical blade may vary along the length of the drum. In particular the pitch may increase from the inlet to the outlet such that the volume of the chambers between the turns also increases from the inlet to the outlet.

A plurality of mixing devices 218 are provided on the surface of the helical blade 214 at the outer edge thereof. There are multiple mixing devices 218 for each turn of the helical blade 214, and as shown in this example there can be eight for each turn of the helical blade 214.

The mixing devices 218 and the helical blade 214 can be seen more clearly in FIG. 10 where the cylindrical tube 212 is removed for clarity. FIG. 10 also shows pipework used to supply fluid to the mixing device 218, including central supply pipes 220 and branch pipes 222 extending to each individual mixing device 218. Central supply pipes 220 may connect to a rotary valve (not shown) for directing fluid into the drum during rotation of the drum.

The mixing devices 218 will now be described in greater detail with reference to FIG. 11 and FIG. 12. FIG. 11 shows a part of two turns of the helical blade 214 in enlarged view with one of the mixing devices 218 at the top of the figure shown in partial section view. FIG. 12 shows a close-up of the top of FIG. 11 so that further detail can be seen. Each of the mixing devices 218 comprises a wedge shaped mixing vane and fluid inlets. The mixing vane in this example has a side profile of the shape of a right-angled triangle with one surface of the triangle being coupled to the surface of the helical blade 214, a vertical surface of the triangle extending at right angles from the surface of the helical blade 214 and a ramp surface of the triangle providing the mixing vane surface. The ramp surface of the triangle extends from a leading edge at the narrow point of the triangle to a trailing edge at the apex of the triangle that is furthest from the helical blade 214. The trailing edge of the mixing vane is provided with fluid inlets 24 which convey fluid supplied via the pipes 220 and branch pipes 222 through the mixing device 218 and out of the inlets 24 into the rotating drum.

The use of such a rotating drum in the demineralisation process will now be described in greater detail. Advantageously, the demineralisation process is carried out with the rotating drum operating in a “flooded” mode. Here, the level of the reactants is above the top of the helical blade 214, and so the acid solution will overflow from each of the separate volumes 216 and be able to flow from the inlet to the outlet of the drum through the open channel at the centre of the drum. However, the solid portion of the organic feedstock (comprising mineralised tissues such as bones, and/or other animal parts such as fish scale, hooves and the like) will sink to the base of the drum so that the solid parts remain in their separated volumes between the walls of the blade. The acid within the chambers of the drum will therefore overflow to the subsequent chambers in the direction of the outlet causing an increase in the flow of acid solution in the direction of the outlet of the drum. This enables a delivery of fresh acid to the chambers of the drum towards the outlet. The flow of acid solution aids in the replenishment of consumed acid in the chambers towards the outlet of the drum and hence maintains the demineralisation reaction rate along the length of the drum.

The residence time of the mineralised tissue is unaffected by the use of the flooded design but instead remains controlled by the rotation of the drum. By modifying the process to replenish reactive acid along the length of the drum, the demineralisation can be completed in a shorter time and correspondingly over a shorter length of the drum. The drum can consequently be made smaller.

Advantageously, the ratio of acid solution to mineralised tissue increases along the length of the drum from the drum inlet to the drum outlet. The progressive increase in acid allows for the demineralisation rate to be controlled. For example, acid consumed via the demineralisation process occurring in chambers towards the inlet of the drum can be replenished as the reactants are conveyed towards the outlet of the drum to provide a more constant volume of reactive acid along the length of the drum and therefore approach a more constant demineralisation rate throughout the process. The addition can be empirically optimized for a given raw material and equipment configuration. By modifying the process to increase the ratio of acid solution along the length of the drum, and thereby increasing the rate of reaction relative to keeping the ratio constant, the demineralisation can be completed in a shorter time and correspondingly over a shorter length of the drum. The drum can consequently be made smaller.

In some embodiments, the pitch of the helical blade increases along the length of the drum, from the drum inlet towards the drum outlet. Each chamber therefore increases in volume towards the outlet of the drum. Since the mineralised tissue remains at the base of the drum and separated by the walls of the blade, the volume of tissue within each chamber will remain constant as the tissue progresses through the drum. However given that all the chambers will be filled to the same level, the volume of acid solution within each chamber increases along the length of the drum as the pitch of the helical blade increases in correspondence with the increase in volume of the chamber. The ratio of acid solution to mineralised tissue therefore increases along the length of the drum. This has the advantage of increasing the presence of reactive acid compared to consumed acid and therefore aids in the maintenance of a desired reaction rate.

FIGS. 13 and 14 show schematically components suitable for use as a diverter 300 to separate a flow of mineralised tissue remnants into two flows. Here, the diverter 300 is provided by the combination of a screw conveyor 310 and a rotary dosing valve 320. The mineralised tissue remnants are moved along the screw conveyor 310 with the rotary dosing valve 320 attached below. A portion of the mineralised tissue remnants is diverted out of the main flow by the rotary dosing valve 320, while the remainder of the mineralised tissue remnants are transported further by the conveyor 310. The diverter 300 thus splits the flow of mineralised tissue remnants into two portions in a non-selective way. The relative size of the two split flows is set by the rotational speed of the rotary dosing valve 320.

As shown in FIG. 14, the rotary dosing valve 320 comprises an inlet 321 and an outlet 325. Between these is provided the valve chamber, in which is provided a plurality of vanes 322 extending from a rotatable hub 323. The plurality of vanes split the valve chamber into a plurality of pockets 324. The faster the vanes 322 rotate about the hub 323, the faster material moves form the inlet 321 to the outlet 325; in other words, a larger portion of material can be diverted out of the main flow.

Details of the inventions will now be described in further detail in the non-limiting Examples below.

### EXAMPLE 1—DEMINERALISATION OF MINERALISED TISSUE REMNANTS

Salmon bones were hydrolysed, and the solid residue (mineralised tissue remnants) was analysed. It was found to that 100 g of salmon bone residue from hydrolysis contains 31.5 g ash and 18.5 g protein.

100 g salmon bone residue from hydrolysis (mineralised tissue remnants) was combined with aqueous HCl in the proportions listed in the Table 1. The reaction was carried out for the times listed in Table 1, and at the specified temperatures. The remaining solids were collected by vacuum filtration and rinsed with 500 mL tap water. The solids were freeze-dried before analysis, and were analysed to determine the remaining mineral content.

The data demonstrates that the mineralised tissue remnants collected after hydrolysis of salmon bones can effectively be demineralised using hydrochloric acid. For example, in Run 6 (60 min reaction time, 40° C., 3% HCl, 1 part bones to 5 parts acid solution), over 90% ash was removed.

### EXAMPLE 2—DEMINERALISATION OF BONE AND MINERALISED TISSUE REMNANTS

Bones and 3% aq. HCl were combined in the amounts listed in Table 2 and heated at the time and temperatures listed. The mixtures were then vacuum filtered, and the collected solids were freeze-dried before analysis.

Salmon bones are bone residue after enzymatic hydrolysis of salmon cutoffs. Cod bones were produced by steeping cod frames in hot water, then mechanically removing muscle then rinsing with tap water. The remaining bones were milled (to approximately 3×6 mm particle size) then freeze-dried.

The data demonstrates that minerals can be effectively removed from cod bones and poultry bones, as well as from mineralised tissue remnants from enzymatic hydrolysis of salmon cutoffs.

### EXAMPLE 3—DEMINERALISATION OF BONE IN ROTATING DRUM PILOT STUDIES

After hydrolysis of salmon bones in the rotating drum, the mineralised tissue remnants (bones) were separated from the other hydrolysis fractions. Thereafter, the bones were again fed into the rotating drum, together with 3% HCl, in order to undergo demineralisation for 2 hours. The differences in the content of amino acids, dry matter, ash, crude protein and fat between the mineralised bones (starting material), and after 2 hours of demineralisation (the demineralised tissue remnants, i.e. ossein) are shown in Table 3. The ash content was reduced from 31.9% in the mineralised bones (starting material) to 3.5% in the demineralised tissue remnants (ossein) after 2 hours of demineralisation. Concomitantly, the relative crude protein content increased to 23.7% in the demineralised tissue remnants (ossein) compared to 18.8% in the starting material (on a wet weight basis). The relative increase in crude protein after demineralisation was confirmed by amino acid analysis, showing an average 2.4 increase (on dry matter basis) in the content of amino acids in the demineralised tissue remnants (ossein) as compared to in the mineralised bones.

### EXAMPLE 4—ENZYMATIC HYDROLYSIS OF DEMINERALISED TISSUE REMNANTS

200 g of ossein (acid-treated salmon bone fragments) and 600 g tap water were combined in a glass reactor. The pH was 3.4. To this, 18.0 mL 3.0 M NaOH (aq) was added to adjust the pH to 6. The mixtures were heated to 55° C. (heating time approximately 12 minutes) and enzymes were added as listed in Table 4. The mixtures were stirred at 55° C. for 60 or 120 minutes. The mixtures were then heated to 90° C. in a microwave oven, and held at that temperature for 10 minutes to denature the enzymes. After cooling to room temperature in an ice bath, the remaining solids were collected by vacuum filtration (Whatman 589/1 filter). The mass of the filter cake and filtrate were recorded. The solids were freeze-dried and weighed again before analysis. The filtrates were analysed without further purification or concentration.

For comparison, 76.62 g starting material (ossein) was freeze-dried, yielding 24.19 g dry material, containing 6.4% hydroxyproline, 86.1% protein (N×6.25), and 98.5% dry matter. Thus 200 g ossein starting material contains 63.2 g dry matter, 54.5 g protein, and 4.04 g hydroxyproline.

The data shows that the yield of soluble protein after hydrolysis of ossein is around 40%, and all ossein protein can be recovered.

### EXAMPLE 5—ENZYMATIC HYDROLYSIS OF MINERALISED TISSUE REMNANTS

Salmon cutoffs were stored frozen for around 2 months then ground using a meat grinder immediately before use. 250 g ground salmon cutoffs, 6.25 g ossein (acid-treated bone residues), and 250 g tap water were combined in a glass reactor. The mixtures were heated to 55° C. (heating time around 12 minutes) and enzymes were added as described in the table. The mixtures were stirred at 55° C. for 60 or 120 minutes. The reactions were then heated to 90° C. in a microwave oven. After cooling to room temperature in an ice bath, the mixtures were centrifuged (20 000×g, 25 min). The liquid fraction was decanted from the sediment, and the mass of wet sediment recorded. The aqueous and oil layers were separated in a separatory funnel. The oil fraction was discarded and the mass of the aqueous fraction was recorded. The aqueous fraction was filtered through a coffee filter to remove small amounts of sediment then stored frozen until analysis. The sediments were freeze-dried before analysis. The results for analysis of the aqueous fraction are shown in Table 5a, whereas the results for analysis of the sediment fraction are shown in Table 5b.

### EXAMPLE 6—ROTATING DRUM

### Basic Example Data:

Capacity: Approx 30 m³ per hour (15 tons raw material and 15 tons of water)
Processing time: 1 hour
Density: 1000 kg/m³
Drum diameter: 3.5 m
Drum diameter inner opening: 1 meter
Drum length: 11.75 m
Pitch of helical blade: 0.375 m

Calculation by means of “Solidworks” show that an outer diameter of 3.5 m, length 11.75 m, inner opening 1 m and 15 cm between liquid level and top of the screw blades—exclusive of the volume of screw blades and mixing vanes—gives a total liquid volume of 30,421 litre.

The incline of the screw is linked to the rotational speed of the drum. High incline gives few “chambers” resulting in a more “batch-like” process. An example configuration (present example) with 750 mm between the vanes with a rotation of ¼ revolution/min gives a periphery speed of 0.0458 m/s.

Nozzles are integrated within each vane as fluid inlets to supply fluid to the rotating drum during mixing. The angled vane propels particles away from the surface of the screw blade, the screw blade continues to rotate and the particles are “launched” from the trailing edge of the vane, whereafter there is turbulent mixing. When the next vane meets the material in the drum the particles in the material would be close to the screw blade again (to be calculated depending on space between screw blade and vane size in each case). By having nozzles along the edge of the vane a very effective mixing is promoted, since the fluid is injected into the zone of turbulent mixing

The vanes in this example could have a height of 500 mm with nozzles mounted 50, 150, 250, 350 and 450 mm from the outer wall of the drum. The maximum height of liquid in the drum will be 1.1 m, but the vane height is 500 mm rather than the full extent of the screw blade or the liquid level since particles will aggregate near the bottom of the rotating drum.

One nozzle typically delivers 10 litre per hour. Having 8 vanes with 5 nozzles each per revolution give a total of (8×15×10)=600 nozzles. Active nozzles (activated when submerged only) will constitute 38%—that is 600×0.38=228 active nozzles a run of one hour.

If we anticipate nozzles ejecting 10 l/hour, which means that the addition of water will be 228×10=2280 I/hour. This gives a volume increase of 7.6%—or increase in liquid level of approximately 6 cm for the chambers at the outlet end of the drum compared to at the inlet end. A steady level could be obtained by a slight and steady increase of the pitch of the screw blade along the length of the drum.

### EXAMPLE 7—ACID TREATMENT OF BONES

100 g salmon bone fragments (recovered from enzymatic hydrolysis of ground salmon cutoffs and stored frozen) were combined with 500 g aqueous acid solutions as shown in Table 6. The mixtures were stirred vigorously at 40° C. for 2 hours. The mixtures were vacuum filtered through Whatman 589/1 filter paper and the filter cake washed with three 300 mL portions of water. When no more water came through the filter, the samples were collected, weighed, and submitted for analysis. As shown in Table 6, HCL was—in a dose-dependent manner—the most efficient acid to remove minerals from the bone proteins (ossein).

### COMPARATIVE EXAMPLE—ATTEMPTING TO RECOVER PROTEIN FROM BONES BY ENZYMATIC HYDROLYSIS WITHOUT DEMINERALISATION

The aim of this Example was to test whether protein (collagen) in salmon bones (which has undergone a first hydrolysis process) could be solubilised by P648L (a protease), by a second hydrolysis process without performing demineralisation.

### Conditions for Second Hydrolysis:

pH=natural (no adjustment)
Temperature: 70° C.
Water/salmon raw material ratio: 1.35
Protease solution to bones ratio: 0.7 mL P648L/100 g bones (7000 ppm)
Hydrolysing time: 16 hrs

Two parallel experiments were carried out:
1. Hydrolysed salmon bones—no addition of enzyme (salmon blank)
2. Hydrolysed salmon bones—added P648L

The results are shown in Table 7. Protein yield in the aqueous fraction after the second hydrolysis was 27% (rather low). From Table 4, the corresponding yield from demineralised bones using the same enzyme (P648, exp 1&2) was 37-43%—but the experiments shown in Table 4 used less protease. So protein recovery is improved for demineralised bones, compared to mineralised bones, even when using less protease (2000 ppm).

TABLE 1

| | | | | | | | | | | % ash |
|---|---|---|---|---|---|---|---|---|---|---|
| | | | | | Bone to | mass freeze- | dry | | | removed |
| | time | temp | | 37% HCl_{(aq)} | acid + water | dried filter | matter | Ash | Remaining | from starting |
| Run | (min) | (° C.) | HCl (%) | (g) | ratio | cake (g) | (wt %) | (wt %) | Ash (g) | material |
| 1 | 60 | 40 | 2.0 | 27.0 | 1 to 5 | 28.8 | 97.1 | 42.4 | 12.2 | 61.2 |
| 2 | 120 | 40 | 2.0 | 27.0 | 1 to 5 | 27.39 | 97.5 | 41.4 | 11.3 | 64.0 |
| 3 | 60 | 55 | 2.0 | 27.0 | 1 to 5 | 27.42 | 97.3 | 42.3 | 11.6 | 63.2 |
| 4 | 120 | 55 | 2.0 | 27.0 | 1 to 5 | 29.43 | 97.3 | 43.2 | 12.7 | 59.6 |
| 5 | 60 | 40 | 3.0 | 40.5 | 1 to 5 | 18.03 | 97.7 | 14.0 | 2.5 | 92.0 |
| 6 | 120 | 40 | 3.0 | 40.5 | 1 to 5 | 18.54 | 97.8 | 12.6 | 2.3 | 92.6 |
| 7 | 60 | 55 | 3.0 | 40.5 | 1 to 5 | 19.11 | 97.6 | 17.8 | 3.4 | 89.2 |
| 8 | 120 | 55 | 3.0 | 40.5 | 1 to 5 | 18.04 | 97.6 | 16.6 | 3.0 | 90.5 |
| 9 | 60 | 40 | 2.0 | 16.2 | 1 to 3 | 39.4 | 97.4 | 53.5 | 21.1 | 33.0 |
| 10 | 120 | 40 | 2.0 | 16.2 | 1 to 3 | 40.44 | 97.2 | 53.3 | 21.6 | 31.5 |
| 11 | 60 | 55 | 2.0 | 16.2 | 1 to 3 | 40.8 | 97.3 | 53.0 | 21.6 | 31.3 |
| 12 | 120 | 55 | 2.0 | 16.2 | 1 to 3 | 41.87 | 97.8 | 53.4 | 22.4 | 29.0 |
| 13 | 60 | 40 | 3.0 | 24.3 | 1 to 3 | 33.33 | 97.1 | 45.0 | 15.0 | 52.4 |
| 14 | 120 | 40 | 3.0 | 24.3 | 1 to 3 | 34.69 | 97.5 | 45.3 | 15.7 | 50.1 |
| 15 | 60 | 55 | 3.0 | 24.3 | 1 to 3 | 35.23 | 97.4 | 46.0 | 16.2 | 48.5 |
| 16 | 120 | 55 | 3.0 | 24.3 | 1 to 3 | 36.2 | 97.4 | 45.8 | 16.6 | 47.3 |

TABLE 2

| | | | | | | | | | mass |
|---|---|---|---|---|---|---|---|---|---|
| | | | | | | | | | freeze- |
| | | | dry | | protein | | | | dried |
| | | mass | matter | ash | (N × 6.25) | protein | time | temp | filter |
| Run | Bone | (g) | (wt %) | (wt %) | wt % | (g) | (min) | (° C.) | cake (g) |
| 1 | Salmon | 100.0 | 52.3 | 31.5 | 18.5 | 18.5 | 120 | 55 | 18.92 |
| 2 | Salmon | 100.0 | 52.3 | 31.5 | 18.5 | 18.5 | 120 | 55 | 17.92 |
| 3 | Cod* | 100.0 | 97.6 | 63.1 | 31.3 | 31.3 | 120 | 55 | 72.35 |
| 4 | Cod* | 100.0 | 97.6 | 63.1 | 31.3 | 31.3 | 120 | 55 | 71.24 |
| 5 | Poultry | 50.0 | 49.0 | 28.2 | 18.0 | 9.0 | 120 | 55 | 6.2 |
| 6 | Poultry | 50.0 | 49.0 | 28.2 | 18.0 | 9.0 | 60 | 55 | 3.85 |
| 7 | Poultry | 50.0 | 49.0 | 28.2 | 18.0 | 9.0 | 60 | 40 | 4.75 |

| | | | | | | | % |
|---|---|---|---|---|---|---|---|
| | | | Remaining | | Remaining | % ash | protein |
| | dry | protein | protein in | | Ash in | removed | removed |
| | matter | (N × 6.25) | sediment | Ash | sediment | from | from |
| Run | (wt %) | wt % | (g) | (wt %) | (g) | bones | bones |
| 1 | 97.9 | 76.5 | 14.47 | 19.9 | 3.8 | 88.0 | 21.9 |
| 2 | 97.9 | 81.4 | 14.59 | 17.7 | 3.2 | 89.9 | 21.3 |
| 3 | 97.6 | 36.9 | 26.70 | 57.2 | 41.4 | 34.4 | 14.2 |
| 4 | 97.7 | 38 | 27.07 | 57.3 | 40.8 | 35.3 | 13.0 |
| 5 | 98.6 | 72.8 | 4.51 | 15.9 | 1.0 | 93.0 | 49.8 |
| 6 | 99.1 | 81 | 3.12 | 9.1 | 0.4 | 97.5 | 65.3 |
| 7 | 98.6 | 81.9 | 3.89 | 11.7 | 0.6 | 96.1 | 56.7 |

TABLE 3

| | | Ossein | Per dry matter | | Fold change after |
|---|---|---|---|---|---|
| Amino acid | Bone | 2 hr | Bone | Ossein 2hr | 2 hr acidic |
| Asx | 1.4 | 1.6 | 2.6 | 6.0 | 2.3 |
| Glx | 2 | 2.5 | 3.8 | 9.4 | 2.5 |
| OH-Pro | 1.5 | 1.8 | 2.8 | 6.8 | 2.4 |
| Ser | 0.9 | 1.1 | 1.7 | 4.2 | 2.4 |
| Gly | 4.5 | 5.5 | 8.5 | 20.8 | 2.4 |
| His | 0.45 | 0.58 | 0.9 | 2.2 | 2.6 |
| Arg | 1.4 | 1.7 | 2.6 | 6.4 | 2.4 |
| Thr | 0.54 | 0.71 | 1.0 | 2.7 | 2.6 |
| Ala | 1.6 | 2.1 | 3.0 | 7.9 | 2.6 |
| Pro | 2 | 2.4 | 3.8 | 9.1 | 2.4 |
| Tyr | 0.29 | 0.36 | 0.5 | 1.4 | 2.5 |
| Val | 0.48 | 0.59 | 0.9 | 2.2 | 2.5 |
| Met | 0.66 | 0.81 | 1.2 | 3.1 | 2.4 |
| Ile | 0.31 | 0.38 | 0.6 | 1.4 | 2.4 |
| Leu | 0.55 | 0.67 | 1.0 | 2.5 | 2.4 |
| Phe | 0.43 | 0.53 | 0.8 | 2.0 | 2.5 |
| Lys | 0.77 | 0.85 | 1.5 | 3.2 | 2.2 |
| Sum Amino | 19.8 | 24.2 | 37.4 | 91.2 | 2.4 |
| acids | | | | | |
| Dry Matter | 52.9 | 26.5 | | | |
| Ash | 31.9 | 3.5 | 60.3 | 13.2 | 0.2 |
| Crude | 18.8 | 23.7 | 35.5 | 89.4 | 2.5 |
| Fat | 1.2 | 0.5 | 2.3 | 1.9 | 0.8 |
| Sum | 51.9 | 27.7 | 98.1 | 105 | |

TABLE 4

| | | | | | | | water- | | |
|---|---|---|---|---|---|---|---|---|---|
| | | | | | | | soluble | | protein |
| | | Enzyme | Enzyme | | mass of | dry | protein - | protein in | recovery |
| | time | dose | dose | | filtrate | matter | kjeldahl | aqueous | in aq. |
| Run | (min) | (ppm) | (mL) | Protease | (g) | (wt %) | (N × 6.25) | phase (g) | phase (%) |
| 1 | 60 | 2000 | 0.4 | P648 | 636.9 | 3.7 | 3.2 | 20.4 | 37.5 |
| 2 | 120 | 2000 | 0.4 | P648 | 602.4 | 4.2 | 3.9 | 23.5 | 43.2 |
| 3 | 60 | 2000 | 0.4 | P950 | 587.5 | 2.9 | 2.8 | 16.5 | 30.2 |
| 4 | 120 | 2000 | 0.4 | P950 | 641.7 | 3.4 | 3 | 19.3 | 35.4 |
| 5 | 60 | 1000 + | 0.2 + | P950L + | 648.9 | 3.8 | 3.3 | 21.4 | 39.4 |
| | | 1000 | 0.2 | PDN | | | | | |
| 6 | 120 | 1000 + | 0.2 + | P950L + | 619.6 | 4 | 3.5 | 21.7 | 39.9 |
| | | 1000 | 0.2 | PDN | | | | | |
| 7 | 60 | 1000 + | 0.2 + | P950 + | 631.5 | 2.9 | 2.3 | 14.5 | 26.7 |
| | | 1000 | 0.2 | P144G | | | | | |
| 8 | 120 | 1000 + | 0.2 + | P950 + | 662.7 | 3.5 | 2.5 | 16.6 | 30.5 |
| | | 1000 | 0.2 | P144G | | | | | |

| | |
|---|---|
| 200 g ossein raw | na |

| | | | mass | mass of | | | | protein | |
|---|---|---|---|---|---|---|---|---|---|
| | | | of wet | freeze- | | protein - | protein | recovery | |
| | | Enzyme | filter | dried | dry | Kjeldahl | remaining | solid in | sum |
| | time | dose | cake | filter | matter | (N × | in solids | phase | protein |
| Run | (min) | (ppm) | (g) | cake (g) | (wt %) | 6.25) | (g) | (%) | recovery |
| 1 | 60 | 2000 | 116.79 | 38.41 | 98.2 | 82.5 | 31.7 | 58.3 | 95.7 |
| 2 | 120 | 2000 | 159.6 | 36.07 | 98.0 | 80.3 | 29.0 | 53.2 | 96.4 |
| 3 | 60 | 2000 | 177.94 | 42.64 | 98.1 | 82.7 | 35.3 | 64.8 | 95.1 |
| 4 | 120 | 2000 | 116.71 | 37.87 | 98.3 | 81.6 | 30.9 | 56.8 | 92.2 |
| 5 | 60 | 1000 + | 119.29 | 42.05 | 98.2 | 81.2 | 34.1 | 62.8 | 102.1 |
| | | 1000 | | | | | | | |
| 6 | 120 | 1000 + | 156.65 | 36.29 | 98.1 | 82.4 | 29.9 | 55.0 | 94.8 |
| | | 1000 | | | | | | | |
| 7 | 60 | 1000 + | 133.93 | 41.49 | 98.3 | 88.1 | 36.6 | 67.2 | 93.9 |
| | | 1000 | | | | | | | |
| 8 | 120 | 1000 + | 117.1 | 39.03 | 98.3 | 84.9 | 33.1 | 60.9 | 91.4 |
| | | 1000 | | | | | | | |

| | | | | | | | |
|---|---|---|---|---|---|---|---|
| 200 g ossein raw | 200 | 63.2 | 98.5 | 86.1 | 54.4 | 100.0 | 100.0 |

TABLE 5a

| | | | | | Aqueous phase | | | | | | |
|---|---|---|---|---|---|---|---|---|---|---|---|
| | | | | | | | | | Hydroxy- | | protein |
| | | Enzyme | Enzyme | | Aq. | dry | protein - | Hydroxy- | proline | protein in | yield in |
| | time | dose | dose | | Phase | matter | Kjeldahl | proline | yield | aq. Phase | aq. Phase |
| Run | (min) | (ppm) | (mL) | Protease | (g) | (wt %) | (N × 6.25) | (wt %) | (%) | (g) | (%) |
| 1 | 60 | 2000 | 0.5 | P648 | 347.5 | 7.3 | 7 | 0.15 | 50.6 | 24.3 | 55.7 |
| 2 | 120 | 2000 | 0.5 | P648 | 356.4 | 7.7 | 7.5 | 0.16 | 55.4 | 26.7 | 61.2 |
| 3 | 60 | 2000 | 0.5 | P950 | 337 | 6.6 | 6.4 | 0.14 | 45.8 | 21.6 | 49.4 |
| 4 | 120 | 2000 | 0.5 | P950 | 341.8 | 7.2 | 6.9 | 0.16 | 53.1 | 23.6 | 54.0 |
| 5 | 60 | 1000 + 1000 | 0.25 + 0.25 | P950L + PDN X95/1 | 333.5 | 6.9 | 6.6 | 0.17 | 55.0 | 22.0 | 50.4 |
| 6 | 120 | 1000 + 1000 | 0.25 + 0.25 | P950L + PDN X95/1 | 342.9 | 7.4 | 7 | 0.16 | 53.3 | 24.0 | 54.9 |
| 7 | 60 | 1000 + 1000 | 0.25 + 0.25 | P950 + P144G | 327.3 | 6.7 | 6.5 | 0.16 | 50.8 | 21.3 | 48.7 |
| 8 | 120 | 1000 + 1000 | 0.25 + 0.25 | P950 + P144G | 332.1 | 7.5 | 7.2 | 0.18 | 58.0 | 23.9 | 54.7 |
| 9* | 60 | 2000 | 0.5 | P648 | 351.6 | 6.6 | 6.6 | 0.14 | 47.8 | 23.2 | 55.3 |
| ossein | | | | | | | | 2.02 | | | |
| raw | | | | | | | | | | | |
| material | | | | | | | | | | | |

TABLE 5b

| | | | | | Sediment wet (g) | | | |
|---|---|---|---|---|---|---|---|---|
| | | | | | | Sediment | | |
| | | Enzyme | Enzyme | | | freeze- | dry | Hydroxy- |
| | time | dose | dose | | Sediment | dried | matter | proline |
| Run | (min) | (ppm) | (mL) | Protease | wet (g) | (g) | (wt %) | (wt %) |
| 1 | 60 | 2000 | 0.5 | P648 | 87.5 | 29.17 | 97.6 | 1.2 |
| 2 | 120 | 2000 | 0.5 | P648 | 75.3 | 25.58 | 98.3 | 1.4 |
| 3 | 60 | 2000 | 0.5 | P950 | 103.3 | 33.15 | 98.5 | 1.5 |
| 4 | 120 | 2000 | 0.5 | P950 | 92.6 | 29.64 | 98.5 | 1.3 |
| 5 | 60 | 1000 + | 0.25 + | P950L + | 109.3 | 34.6 | 95.6 | 1.5 |
| | | 1000 | 0.25 | PDN X95/1 | | | | |
| 6 | 120 | 1000 + | 0.25 + | P950L + | 89.1 | 28.38 | 98.2 | 1.3 |
| | | 1000 | 0.25 | PDN X95/1 | | | | |
| 7 | 60 | 1000 + | 0.25 + | P950 + | 98.7 | 30.68 | 98.6 | 1.1 |
| | | 1000 | 0.25 | P144G | | | | |
| 8 | 120 | 1000 + | 0.25 + | P950 + | 86.1 | 27.52 | 98.3 | 1.4 |
| | | 1000 | 0.25 | P144G | | | | |
| 9* | 60 | 2000 | 0.5 | P648 | 85.5 | 28.56 | 98.5 | 1.4 |
| salmon | | | | | 250 | | 40.3 | 0.36 |
| cutoffs | | | | | | | | |
| ossein | | | | | 6.25 | | 31.1 | 2.02 |

| | Sediment wet (g) | | | | | |
|---|---|---|---|---|---|---|
| | Hydroxy- | Hydroxy- | protein - | protein | protein | |
| | proline | proline | Kjeldahl | in | yield in | sum |
| | yield | yield | (N × | sediment | sediment | protein |
| Run | (g) | (%) | 6.25) | (g) | (%) | yield |
| 1 | 0.350 | 34.1 | 58.4 | 17.0 | 39.0 | 94.6 |
| 2 | 0.358 | 34.9 | 58.4 | 14.9 | 34.2 | 95.4 |
| 3 | 0.497 | 48.5 | 61 | 20.2 | 46.3 | 95.6 |
| 4 | 0.385 | 37.5 | 60.5 | 17.9 | 41.0 | 95.0 |
| 5 | 0.519 | 50.6 | 61.8 | 21.4 | 48.9 | 99.3 |
| 6 | 0.369 | 35.9 | 60.3 | 17.1 | 39.2 | 94.1 |
| 7 | 0.337 | 32.9 | 61.5 | 18.9 | 43.2 | 91.9 |
| 8 | 0.385 | 37.5 | 59.1 | 16.3 | 37.2 | 91.9 |
| 9* | 0.400 | 39.0 | 58.3 | 16.7 | 39.6 | 94.9 |
| salmon | 0.900 | 87.7 | 16.8 | 42.0 | 96.1 | |
| cutoffs | | | | | | |
| ossein | 0.126 | 12.3 | 27.2 | 1.7 | 3.9 | |

TABLE 6

| | | | Raw data | | | | Calculated from raw data | | | | |
|---|---|---|---|---|---|---|---|---|---|---|---|
| | Amount | | mass of | | protein | | protein | ash | Recovered | Recovered | |
| | acid | | filtered | dry | wt % | | wt % dry | wt % dry | protein in | ash in | % of |
| | in | | bones | matter | (N × | ash | matter | matter | solid | solid | ash |
| Acid | water | Abs. Amts | (g) | (wt %) | 6.25) | (wt %) | basis | basis | phase (g) | phase (g) | removed |
| Blank | 0 | | 95.0 | 59.2 | 21.5 | 36.1 | 36.3 | 61.0 | 20.4 | 34.3 | |
| (water) | | | | | | | | | | | |
| H₂SO₄ | 2.5% | 12.5 g conc H₂SO₄ | 112.0 | 47.1 | 18.8 | 27.2 | 39.9 | 57.7 | 21.1 | 30.5 | 10.0 |
| HCl | 2% | 27 g 37% HCl | 74.0 | 45.6 | 26.3 | 19.2 | 57.7 | 42.1 | 19.5 | 14.2 | 58.0 |
| HCl | 2.5% | 33.8 g 37% HCl | 69.2 | 40.1 | 27.5 | 12.6 | 68.6 | 31.4 | 19.0 | 8.7 | 74.2 |
| HCl | 3% | 40.5 g 37% HCl | 66.0 | 34.4 | 29.4 | 6.3 | 85.5 | 18.3 | 19.4 | 4.2 | 87.7 |
| Citric acid | 1 g/L | 1 g 50% citric acid | 89.5 | 62.3 | 22.4 | 37.3 | 36.0 | 59.9 | 20.0 | 33.4 | 1.4 |
| | | sol'n | | | | | | | | | |
| Citric acid | 2 g/L | 2 g 50% citric acid | 93.0 | 60.6 | 22.2 | 36.3 | 36.6 | 59.9 | 20.6 | 33.8 | 0.3 |
| | | sol'n | | | | | | | | | |
| Citric acid | 3 g/L | 3 g 50% citric acid | 92.2 | 61.1 | 22.6 | 36.1 | 37.0 | 59.1 | 20.8 | 33.3 | 1.7 |
| | | sol'n | | | | | | | | | |
| Blank | 0 | | 94.8 | 59.2 | 21.2 | 35.2 | 35.8 | 59.5 | 20.1 | 33.4 | |
| (water) | | | | | | | | | | | |
| H₂SO₄ | 2.5% | 12.5 g conc H₂SO₄ | 102.0 | 49.1 | 19.2 | 28.5 | 39.1 | 58.0 | 19.6 | 29.1 | 14.1 |
| HCl | 2% | 27 g 37% HCl | 70.7 | 47.1 | 26.6 | 19.5 | 56.5 | 41.4 | 18.8 | 13.8 | 59.3 |
| HCl + citric | 2% + | 27 g 37% HCl + 1 g | 63.7 | 45.6 | 28.4 | 16.8 | 62.3 | 36.8 | 18.1 | 10.7 | 68.4 |
| acid | 1 g/L | 50% citric acid | | | | | | | | | |
| HCl + | 2% + | 2 g 50% citric | 65.1 | 40.6 | 27.7 | 13 | 68.2 | 32.0 | 18.0 | 8.5 | 75.0 |
| Na2EDTA | 30 g/L | acid + 15 g EDTA | | | | | | | | | |
| HCl + citric | 2% + | 27 g 37% HCl + | 67 | 41.4 | 23.1 | 14.1 | 55.8 | 34.1 | 15.5 | 9.4 | 72.1 |
| acid + | 1 g/L + | 2 g 50% citric | | | | | | | | | |
| Na2EDTA | 30 g/L | acid + 15 g EDTA | | | | | | | | | |
| Citric | 1 g/L + | 1 g 50% citric | 73.4 | 57.6 | 23.8 | 31.4 | 41.3 | 54.5 | 17.5 | 23.0 | 31.9 |
| acid + EDTA | 30 g/L | acid + 15 g EDTA | | | | | | | | | |
| EDTA | 30 g/L | 15 g EDTA | 70.1 | 59.5 | 24.8 | 33 | 41.7 | 55.5 | 17.4 | 23.1 | 31.7 |

TABLE 7

| | | | | | | | | | freeze- | dry | | | | | |
|---|---|---|---|---|---|---|---|---|---|---|---|---|---|---|---|
| | | | | | | | | | dried | matter | crude | | | | |
| | | wet | dry | dry | | P648L | | t = | product | (after | protein | | | | protein |
| | raw | mass | matter | mass | H₂O | enzyme | t = 0 | 17 h | mass | freeze- | (N × 6.25) | ash | fat | protein | recovery |
| Rxn | material | (g) | (%) | (g) | (mL) | (mL) | pH | pH | (g) | drying) | (Dumas) | (%) | (%) | (g) | (%) |
| 1 | salmon bones | 200 | 56.23 | 112.46 | 260 | 0 | 6.8 | 6.5 | — | 98.6 | 36.51 | 59.93 | 1.72 | 41.64 | — |
| | aq phase | — | — | — | — | — | — | — | 1.96 | | 75.9 | | | 1.50 | 3.60 |
| | solid phase | — | — | — | — | — | — | — | 109.98 | 99.10 | 34.8 | 60.5 | | 38.62 | 92.74 |
| 2 | salmon bones | 200 | 56.23 | 112.46 | 260 | 1.4 | 6.8 | 6.2 | — | 98.6 | 36.51 | 59.93 | 1.72 | 41.64 | — |
| | aq phase | — | — | — | — | — | — | — | 12.01 | | 93.3 | | | 11.32 | 27.19 |
| | solid phase | — | — | — | — | — | — | — | 96.96 | 99.00 | 28.4 | 66.5 | | 27.81 | 66.79 |

1. A method of processing an organic feedstock comprising mineralised tissues, the method comprising:
carrying out enzymatic hydrolysis of the organic feedstock to produce hydrolysis fractions and mineralised tissue remnants;
separating the mineralised tissue remnants from the hydrolysis fractions;
demineralising the mineralised tissue remnants to produce solubilised minerals and demineralised tissue remnants.

2. The method of claim 1, wherein the organic feedstock comprises flesh and bones from aquatic animal origin, or terrestrial animal origin.

3. The method of claim 1 or 2, wherein the processing of the organic feedstock is carried out as a continuous process.

4. The method of any preceding claim, wherein demineralising the mineralised tissue remnants comprises treating them with an acid solution in order to solubilise the minerals.

5. The method of claim 4, wherein the acid solution comprises hydrochloric acid.

6. The method of claim 4 or 5, wherein the acid solution comprises acid at a concentration of between 1 and 5 wt %.

7. The method of claim 4, 5 or 6 wherein the weight ratio of mineralised tissue remnant to acid solution is between 1:2 and 1:8.

8. The method of any of claims 4 to 7, wherein demineralising the mineralised tissue remnants comprises progressive addition of acid for controlling the demineralisation rate.

9. The method of any preceding claim, wherein demineralisation of the mineralised tissue remnants is carried out for between 30 to 180 minutes.

10. The method of any preceding claim, wherein the demineralisation of the mineralised tissue remnants is carried out at a temperature between 30 and 70 degrees centigrade.

11. The method of any preceding claim, further comprising separating the solubilised minerals from the demineralised tissue remnants.

12. The method of claim 11, further comprising recovering the solubilised minerals by neutralising the solution of solubilised minerals, optionally using NaOH or KOH, and then processing the neutralised solution, optionally by filtering or centrifuging, to form a mineral slurry.

13. The method of claim 11 or 12, wherein separating the mineralised tissue remnants from the hydrolysis fractions is carried out using a sieve, a dewatering screw press, a filter, or a density separator.

14. The method of any preceding claim, further comprising separating the hydrolysis fractions into: an aqueous fraction comprising water with dissolved protein, polypeptides and amino acids; a sediment fraction comprising insoluble proteins; and an oil fraction.

15. The method of any preceding claim, further comprising carrying out a second enzymatic hydrolysis process on a portion of the demineralised tissue remnants, wherein the second enzymatic hydrolysis process optionally comprises use of a protease.

16. The method of claim 15, comprising separating an output of the second enzymatic hydrolysis process into a sediment fraction and an aqueous fraction, and optionally at least partially drying the sediment fraction and/or aqueous fraction.

17. The method of any preceding claim, further comprising recycling a portion of the demineralised tissue remnants back into the organic feedstock for enzymatic hydrolysis.

18. The method of any preceding claim, comprising drying a portion of the demineralised tissue remnants, and/or drying a portion of the mineralised tissue remnants.

19. The method of any preceding claim, wherein the enzymatic hydrolysis of the organic feedstock and/or demineralising the mineralised tissue remnants takes place in a rotating drum reactor, wherein the rotating drum reactor comprises:
a drum which is rotatable about a central longitudinal axis of the drum, optionally wherein the central longitudinal axis is substantially horizontal;
an inlet at a first point on the drum;
a screw within the drum, wherein the screw includes a helical blade extending along the length of the drum with the outer edge of the helical blade being fixed to the inner surface of the drum such that material can be conveyed and mixed by the helical blade as the drum rotates;
an outlet at a second point along the drum.

20. The method of any preceding claim, further comprising:
grinding the organic feedstock prior to carrying out the enzymatic hydrolysis.

21. A processing plant for processing an organic feedstock comprising mineralised tissue, the processing plant comprising:
a first reaction vessel configured to perform enzymatic hydrolysis of the organic feedstock;
a first separator configured to receive the output from the first reaction vessel, for separating mineralised tissue remnants output from the first reaction vessel from hydrolysis fractions output from the first reaction vessel; and
a second reaction vessel configured to perform demineralisation of the mineralised tissue remnants to produce demineralised tissue remnants and solubilised minerals.

22. The processing plant of claim 21, wherein the processing plant is configured to carry out processing of the organic feedstock as a continuous process.

23. The processing plant of claim 21 or 22, comprising a sieve, a dewatering screw press, a filter, or a density separator for separating the mineralised tissue remnants from the hydrolysis fractions.

24. The processing plant of claim 21, 22, or 23, comprising a three-phase separator for separating the hydrolysis fractions into: an aqueous fraction comprising water with dissolved protein, polypeptides and amino acids; a sediment fraction comprising insoluble proteins; and an oil fraction.

25. The processing plant of any of claims 21 to 24, comprising a separator for separating the solubilised minerals from the demineralised tissue remnants.

26. The processing plant of any of claims 21 to 25, comprising a third reaction vessel for carrying out a second enzymatic hydrolysis process on a portion of the demineralised tissue remnants.

27. The processing plant of claim 26, comprising a separator for separating an output of the third reaction vessel into a sediment fraction and an aqueous fraction.

28. The processing plant of any of claims 21 to 27, wherein the first reaction vessel is configured to receive a portion of the demineralised tissue remnants output from the second reaction vessel.

29. The processing plant of any of claims 21 to 28 comprising one or more dryers, wherein the one or more dryers comprise: a mill dryer and/or an evaporator, and/or a spray dryer.

30. The processing plant of any of claims 21 to 29, comprising a diverter configured to split a flow of mineralised tissue remnants or demineralised tissue remnants into a plurality of flows.

31. The processing plant of any claims 21 to 30, wherein at least one of the first, second and third reaction vessels is a rotating drum reactor, wherein the rotating drum reactor comprises:
a drum which is rotatable about a central longitudinal axis of the drum, optionally wherein the central longitudinal axis is substantially horizontal;
an inlet at a first point on the drum;
a screw within the drum, wherein the screw includes a helical blade extending along the length of the drum with the outer edge of the helical blade being fixed to the inner surface of the drum such that material can be conveyed and mixed by the helical blade as the drum rotates; and
an outlet at a second point along the drum.

32. The processing plant of any of claims 21 to 31 configured to carry out the method of any of claims 1 to 20.

33. A method of demineralising mineralised tissues, the method comprising;
mixing the mineralised tissues with an acid solution in order to solubilise the minerals in the mineralised tissues, wherein
mixing the mineralised tissues with the acid solution takes place in a rotating drum reactor, the rotating drum reactor comprising:
a drum which is rotatable about a central longitudinal axis of the drum;
an inlet at a first point on the drum;
a screw within the drum, wherein the screw comprises a helical blade extending along the length of the drum with the outer edge of the helical blade being fixed to the inner surface of the drum such that material can be conveyed and mixed by the helical blade as the drum rotates; and
an outlet at a second point along the drum.

34. The method of claim 33, wherein the method is carried out as a continuous process.

35. The method of claim 33 or 34, wherein the axis of rotation of the drum is broadly horizontal.

36. The method of claim 33, 34 or 35, wherein an inner edge of the screw blade is attached to an inner cylindrical body, optionally in a watertight fashion.

37. The method of claim 33, 34 or 35, wherein the helical blade extends from the inner surface of the drum toward to the centre of the drum but does not extend along the entire diameter of the drum such that an open channel extends along the axial length of the drum.

38. The method of claim 37, wherein the acid solution fills the rotating drum reactor to above the level of the helical blade, allowing flow of the acid solution along the open channel.

39. The method of any of claims 33 to 38, wherein the ratio of acid solution to mineralised tissue increases along the length of the drum from the inlet to the outlet.

40. The method of any of claims 33 to 39, wherein the pitch of the helical blade increases towards the outlet of the drum.

41. The method of any of claims 33 to 40, wherein acid solution can be introduced to the drum through fluid inlets spaced apart along the axial length of the drum.

42. The method of any of claims 33 to 41, wherein the acid solution comprises hydrochloric acid.

43. The method of any of claims 33 to 42, wherein the acid solution comprises acid at a concentration of between 1 and 5 wt %.

44. The method of any of claims 33 to 43, wherein the weight ratio of mineralised tissue remnants to acid solution is between 1:2 and 1:8.

45. The method of any of claims 33 to 44, wherein demineralisation of the mineralised tissue remnants is carried out for between 30 to 180 minutes.

46. The method of any of claims 33 to 45, wherein the demineralisation of the mineralised tissue remnants is carried out at a temperature between 30 and 70 degrees centigrade.
